(19) United States
(12) Patent Application Publication
(10) Pub. No.: US 2008/0214509 A1
(43) Pub. Date: Sep. 4, 2008

(54) Methods for enhancing the efficacy of vascular disrupting agents

(76) Inventors: Robert Kerbel, Toronto (CA); Yuval Shaked, Toronto (CA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/074,555

(22) Filed: Mar. 3, 2008

Related U.S. Application Data
(60) Provisional application No. 60/904,460, filed on Mar. 2, 2007.

(51) Int. Cl. A61K 31/09; A61K 31/135; A61K 31/661; A61P 35/00

(52) U.S. Cl. 514/130; 514/720; 514/646

(57) ABSTRACT

This invention relates to methods for treating, preventing and/or managing cancer in a subject including enhancing the efficacy of a Vascular Disrupting Agent (e.g., a combretastatin or derivative thereof) by administering to the subject a Chemokine Receptor Antagonist (e.g., a CXCR4 antagonist) or Chemokine Antagonist (e.g., a SDF-1 antagonist) sequentially or simultaneously in combination with said Vascular Disrupting Agent.

### RELATED APPLICATIONS

This application claims priority to U.S. Provisional Application Ser. No. 60/904,460, entitled “Methods for Enhancing the Efficacy of Vascular Damaging Agents”, filed on Mar. 2, 2007. The entire contents of the aforementioned application are hereby incorporated herein by reference.

### BACKGROUND OF THE INVENTION

The National Cancer Institute has estimated that in the United States alone, 1 in 3 people will be struck with cancer during their lifetime. Moreover approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed.

Anticancer agents have been classified based upon their mechanism of action. One promising new class of chemotherapeutic are referred to as a Vascular Disrupting Agents (VDAs) (or alternatively, Vascular Damaging Agents, Vascular Targeting Agents (VTAs) or Anti-vascular agents). The primary mechanism of action of VDAs is “vascular targeting”, in which the neovasculature of solid tumors is selectively disrupted, resulting in a transient decrease or complete shutdown of tumor blood flow that results in secondary tumor cell death due to hypoxia, acidosis, and/or nutrient deprivation (Dark et al., Cancer Res., 57: 1829-34, (1997); Chaplin et al., Anticancer Res., 19: 189-96, (1999); Hill et al., Anticancer Res., 22(3): 1453-8 (2002); Holwell et al., Anticancer Res., 22(2A):707-11, (2002). While effective in killing the vast majority of the tumor mass, some tumors are nonetheless resistant to treatment with VDAs, such as combretastatin A-4 phosphase (CA4P), due to a rim of viable tumor tissue, which can serve to repopulate the tumor, eventually leading to progression of tumor cell growth (Dark et al., Cancer Res., 57: 1829-34, (1997); Chaplin et al., Anticancer Res., 19: 189-96, (1999)).

There is thus an urgent need in the art to provide methods for improving of VDA therapy by preventing tumor regrowth due to endothelial cell mobilization.

### SUMMARY OF THE INVENTION

The present invention provides, in part, methods for producing an enhanced antitumor effect wherein a combination of agents is employed. In particular aspects, the methods of the invention comprise the administration (e.g., sequential administration or co-administration) of a Vascular Disrupting Agent (hereinafter, a “VDA”) and a chemokine receptor antagonist (e.g., a CXCR antagonist) and/or a chemokine antagonist (e.g., an SDF-1 antagonist). The methods of the present invention provide advantages such as greater overall therapeutic efficacy of VDA therapy, for example, by preventing tumor regrowth. Further, where a tumor to be treated is not optimally responsive (e.g. resistant) to treatment with a Vascular Disrupting Agent, use of the present combination therapy methods can nonetheless provide effective treatment.

In one aspect, the invention provides a method for producing an anti-tumor effect in an patient suffering from a cancer or tumor, the method comprising administering to the patient a VDA and a CXCR antagonist (e.g., a CXCR4 antagonist) or chemokine antagonist (e.g., a SDF-1 antagonist). The VDA may be administered at any time relative to administration of said CXCR or chemokine antagonist. In one embodiment, the VDA and CXCR4 (or SDF-1) antagonist may be administered simultaneously to produce a potentiated antitumor effect. In another embodiment the VDA and CXCR4 (or SDF-1) antagonist may be administered sequentially in any order to produce a potentiated antitumor effect. In one preferred embodiment, a CXCR antagonist (e.g. a CXCR-4 antagonist of Formula I) or chemokine antagonist (e.g., a SDF-1 antagonist) is sequentially administered in any order with effective amounts of a VDA (e.g. a combretastatin). In a preferred embodiment, 1,1′-[1,4-phenylenebis(methylene)]-bis-1,4,8,11-tetra-azacyclotetradecane is sequentially administered in any order with an effective amount of a VDA (e.g., a combretastatin). In a still more preferred embodiment, combretastatin A-4 phosphate (CA4P) or combretastatin A-1 diphosphate (CA1P) is sequentially or simultaneously administered in any order with an effective amount of 1,1′-[1,4-phenylenebis(methylene)]-bis-1,4,8,11-tetra-azacyclotetradecane.

In another aspect, the invention provides a pharmaceutical composition comprising a VDA (e.g., a combretastatin) and a CXCR antagonist (e.g. a CXCR4 antagonist of Formula I) or chemokine antagonist (e.g., a SDF-1 antagonist). In a particular embodiment, the CXCR4 antagonist is 1,1′-[1,4-phenylenebis(methylene)]-bis-1,4,8,11-tetra-azacyclotetradecane or a pharmaceutically acceptable salt thereof. In one preferred embodiment, said pharmaceutical composition comprises AMD3100 and CA1P. In another preferred embodiment, said pharmaceutical composition comprises AMD3100 and CA4P.

In another aspect, the pharmaceutical composition can be present in a subtherapeutic dose for one or both individual agents, the agents (i.e., the VDA and CXCR (or chemokine) antagonist) being more effective when used in combination. Alternatively, each agent can be provided at therapeutic doses for one or both individual agents, such as those found in the Physician's Desk Reference.

In another aspect, the present invention further provides pharmaceutical kits. Exemplary kits of the invention comprise a first pharmaceutical composition comprising a CXCR antagonist (e.g., a CXCR4 antagonist, e.g. a compound of the Formula I) or chemokine antagonist (e.g., a SDF-1 antagonist) and a second pharmaceutical composition comprising a VDA (e.g., a combretastatin) together in a package. The CXCR (or chemokine) antagonist and VDA can be present, for example, in a subtherapeutic dose for one or both individual agents, the agents being effective in combination and providing reduced side effects while maintaining efficacy. Alternatively, each agent can be provided at a therapeutic dose, such as those found for the agent in the Physician's Desk Reference.

In certain aspects, the present invention provides methods of administering a VDA, preferably a combretastatin or combretastatin derivative, together with a CXCR or chemokine antagonist in order to potentiate the overall efficacy of the combination. In one embodiment, the VDA and CXCR antagonist (or chemokine antagonist) are administered simultaneously. In other embodiments, the VDA and CXCR antagonist (or chemokine antagonist) are administered sequentially. When administered sequentially, a CXCR (or chemokine) antagonist can preferably be administered, for example, within 24 hours of the administration of the VDA, such as within 1-24 hours prior, 2-24 hours prior, 3-24 hours prior, 6-24 hours prior, 8-24 hours prior, or 12 to 24 hours prior to administration, or such as within 1-24 hours after, 2-24 hours after, 3-24 hours after, 6-24 hours after, 8-24 hours after, or 12 to 24 hours after administration of the VDA.

In other aspects, the invention provides a method for producing an anti-tumor effect in a subject suffering from cancer or a tumor, the method comprising administering to the patient a VDA and a CXCR antagonist (or chemokine antagonist) in amounts effective therefor.

In another aspect, the invention provides a method for preventing tumor regrowth in a subject suffering from cancer or a tumor, the method comprising administering to the patient a VDA and a CXCR antagonist (or chemokine antagonist) in amounts effective therefor.

In yet another aspect, the invention provides a method for inhibiting tumor-associated angiogenesis in a subject that is treated with a VDA, the method comprising administering to the patient a CXCR antagonist (or chemokine antagonist) in amounts effective therefor.

In still another aspect, the invention provides a method for inhibiting homing and retention of circulating endothelial progenitor (CEP) cells or other proangiogenic cells to the tumor of a subject that is treated with a VDA, the method comprising administering to the patient a CXCR antagonist (or chemokine antagonist) in amounts effective therefor.

In certain embodiments of the invention, the CXCR antagonist is a CXCR4 antagonist. In other embodiments, the chemokine antagonist is a SDF-1 antagonist.

In certain embodiments, the VDA is a combretastatin agent. In certain embodiments, the combretastatin agent is a combretastatin derivative of Formula V:

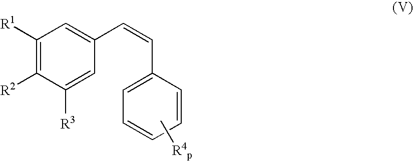

wherein
each of R¹, R² and R³, independently of the others, is selected from the group consisting of hydrogen, C₁₋₆ alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR⁷ substituted with one or more of the same or R⁷ or R⁶, —B(OR⁷)₂, —B(NR⁸R⁸)₂, —(CH₂)ₘ—R⁶, —(CHR⁷)ₘ—R⁶, —O—(CH₂)ₘ—R⁶, —S—(CH₂)ₘ—R⁶, —O—CHR⁷R⁶, —CR⁷(R⁶)₂, —O—(CHR⁷)ₘ—R⁶—O—(CH₂)ₘ—CH[(CH₂)ₘR⁶]R⁶, —S—(CHR⁷)ₘ—R⁶, —C(O)NH—(CH₂)ₘ—R⁶, —C(O)NH—(CHR⁷)ₘ—R⁶, —O—(CH₂)ₘ—C(O)NH—(CH₂)ₘ—R⁶, —S—(CH₂)ₘ—C(O)NH—(CH₂)ₘ—R⁶, —O—(CHR⁷)ₘ—C(O)NH—(CHR⁷)ₘ—R⁶, —S—(CHR⁷)ₘ—C(O)NH—(CHR⁷)ₘ—R⁶, —NH—(CH₂)ₘ—R⁶, —NH—(CHR⁷)ₘ—R⁶, —NH[(CH₂)ₘR⁶], —N[(CH₂)ₘR⁶]₂, —NH—C(O)—NH—(CH₂)ₘ—R⁶, —NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate
each R⁶ is a suitable group independently selected from the group consisting of ═O, —OR⁷, C₁₋₃ haloalkyloxy, —OCF₃, ═S, —SR⁷, ═NR⁷, ═NOR⁷, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(NOH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NHC(O)]ₙOR⁷, —[NR⁷C(O)]ₙOR⁷, —[NHC(O)]ₙNR⁸R⁸, —[NR⁷C(O)]ₙNR⁸R⁸, —[NHC(NH)]ₙNR⁸R⁸ and —[NR⁷C(NR⁷)]ₙNR⁸R⁸;
each R⁷ is independently selected from the group consisting of hydrogen, C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R⁷ or suitable R⁶ groups;
each m independently is an integer from 1 to 3;
each n independently is an integer from 0 to 3;
p is an integer from 1 to 5, and

wherein two adjacent R⁴ groups and their intervening atoms are bonded to form a 5-8 membered ring fused to the central phenyl group.

In a particularly preferred embodiment, the combretastatin agent is a compound of Formula II:

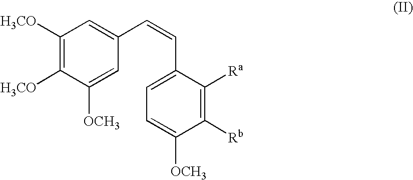

or a pharmaceutically acceptable salt thereof wherein R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and

R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

In a preferred embodiment R^{a} is a phosphate of formula:

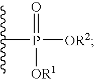

and R^{b} is a phosphate of formula:

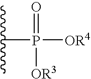

wherein OR¹, OR², OR³ and OR⁴ are each, independently, H, —O⁻QH⁺ or —O⁻M⁺, wherein M⁺ is a monovalent or divalent metal cation, and Q is, independently:

a) an amino acid containing at least two nitrogen atoms where one of the nitrogen atoms, together with a proton, forms a quaternary ammonium cation QH⁺; or

b) an organic amine containing at least one nitrogen atom which, together with a proton, forms a quaternary ammonium cation, QH⁺.

In one embodiment, the combretastatin agent is a compound of Formula IIb:

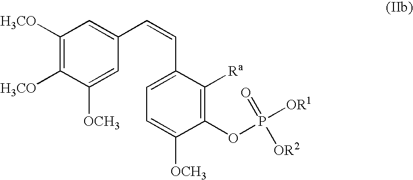

wherein R^{a} is H or OP(O)(OR³)OR⁴; and

OR¹, OR², OR³ and OR⁴ are each, independently, H, —O⁻QH⁺ or —O⁻M⁺, wherein M⁺ is a monovalent or divalent metal cation, and Q is, independently:

a) an amino acid containing at least two nitrogen atoms where one of the nitrogen atoms, together with a proton, forms a quaternary ammonium cation QH⁺; or

b) an organic amine containing at least one nitrogen atom which, together with a proton, forms a quaternary ammonium cation, QH⁺.

In one embodiment, for Formula IIb, R³ is H or OP(O)(OR³)OR⁴, and R¹, R², R³ and R⁴ are each, independently, an aliphatic organic amine, alkali metals, transition metal, heteroarylene, heterocyclyl, nucleoside, nucleotide, alkaloid, amino sugar, amino nitrile, or nitrogenous antibiotic. In another embodiment, for Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine. In another embodiment, Formula II is represented by a compound of Formula III:

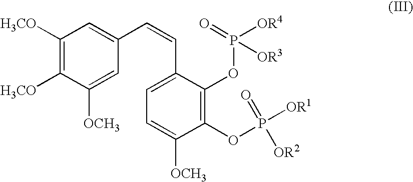

and pharmaceutically acceptable salts thereof.

In certain embodiments of the invention, the combretastatin agent is administered at a dose ranging from between 45 mg/kg and 63 mg/kg.

In other embodiments of the invention, the CXCR4 antagonist is a compound of Formula I or a pharmaceutically acceptable salt or metal complex thereof:

Z-R—Ar—R′—Y (I)

wherein

Z and Y are each, independently, a cyclic polyamine moiety having a total of 9 to 24 atoms and from 2 to 6 optionally substituted nitrogens spaced by two or more optionally substituted carbon atoms from each other, and which may optionally comprise a fused aromatic or heteroaromatic ring;

R and R′ are each, independently, selected from the group consisting of straight, branched, or cyclic C₁₋₆-alkyl groups; and

Ar is an aromatic or heteroaromatic ring, optionally substituted at single or multiple positions with electron-donating or electron-withdrawing groups.

In certain embodiments, for Formula I, Z and Y are each, independently, a cyclic polyamine moiety having a total of 14 to 20 atoms and from 3 to 6 optionally substituted amino nitrogens spaced by two or more optionally substituted carbon atoms from each other. In one embodiment, for Formula I, Ar is phenyl. In another embodiment, for Formula I, R and R′ are CH₂.

In other embodiments, Formula I is represented by 1,1′-[1,3-phenylenebis(methylene)]-bis-1,4,8,11-tetra-azacyclotetradecane; 1,1′-[1,4-phenylenebis(methylene)]-bis-1,4,8,11-tetra-azacyclotetradecane; 1,1′-[3,3′-biphenylene-bis-(methylene)]-bis-1,4,8,11-tetraazacyclotetradecane; 11,11′-[1,4-phenylene-bis-(methylene)]-bis-1,4,7,11-tetraazacyclotetradecane; 1,11′-[1,4-phenylene-bis-(methylene)]-1,4,8,11-tetraazacyclotetradecane-1,4,7,11-tetraazacyclotetradecane; 1,1′-[2,6-pyridine-bis-(methylene)]-bis-1,4,8,11-tetraazacyclotetradecane; 1,1-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[5-nitro-1,3-phenylenebis(methylene)]bis-1,4,8,11-tetraazacyclotetradecane; 1,1′-[2,4,5,6-tetrachloro-1,3-phenyleneis(methylene)]-bis-1,4,8,11-tetraazacyclotetradecane; 1,1′-[2,3,5,6-tetra-fluoro-1,4-phenylenebis(methylene)]bis-1,4,8,11-tetraazacyclotetradecane; 1,1′-[1,4-naphthylene-bis-(methylene)]bis-1,4,8,11-tetraazacyclotetradecane; 1,1′-[1,3-phenylenebis-(methylene)]bis-1,5,9-triazacyclododecane; 1,1′-[1,4-phenylene-bis-(methylene)]-1,5,9-triazacyclododecane; 1,1′-[3,3′-biphenylene-bis-(methylene)]-bis-1,4,8,11-tetraazacyclotetradecane; 1,1′-[2,6-pyridine-bis-(methylene)]-bis-1,4,8,11-tetraazacyclotetradecane; 1,1′-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[2,5-dimethyl-1,4-phenylenebis-(methylene)]-bis-1,4,8,11-tetraazacyclotetradecane; 1,1′-[2,5-dichloro-1,4-phenylenebis-(methylene)]-bis-1,4,8,11-tetraazacyclotetradecane; 1,1′-[2-bromo-1,4-phenylenebis-(methylene)]-bis-1,4,8,11-tetraazacyclotetradecane; or 1,1′-[6-phenyl-2,4-pyridinebis-(methylene)]-bis-1,4,8,11-tetraazacyclotetradecane; and pharmaceutically acceptable salts thereof.

In certain embodiments of the invention, the cancer is selected from the group consisting of ovarian cancer, fallopian tube cancer, cervical cancer, breast cancer, lung cancer, melanoma, and primary cancer of the peritoneum. In other embodiments, the tumor is a solid tumor selected from the group consisting of a melanoma, an ovarian tumor, a cervical tumor, a breast tumor, small cell lung tumor, a non-small cell lung tumor, a fallopian tube tumor, and a primary tumor of the peritoneum.

In certain embodiments, the invention provides a method of treating a tumor in a subject in need thereof by administering to the subject a pharmaceutical composition comprising a compound of the Formula I and a compound of the Formula II or IIb wherein the compound of Formula I is administered first followed by administration of a compound of Formula II or IIb.

In other embodiments, the invention provides a method of treating a tumor in a subject in need thereof by administering to the subject a pharmaceutical composition comprising a compound of the Formula I and a compound of the Formula II or IIb, wherein the compound of Formula II or IIb is administered first followed by administration of a compound of Formula I.

In yet other embodiments, the invention provides a method of treating a tumor in a subject in need thereof by administering to the subject a pharmaceutical composition comprising a compound of the Formula I and a compound of the Formula II or IIb, wherein the compound of Formula I and a compound of Formula II or IIb are administered simultaneously.

In one preferred embodiment, the invention provides a method of treating a tumor in a subject in need thereof by administering to the subject a pharmaceutical composition comprising a AMD3100 and CA1P. In another preferred embodiment, the invention provides a method of treating a tumor in a subject in need thereof by administering to the subject a pharmaceutical composition comprising AMD3100 and CA4P.

In certain embodiments, the subject is a mammal. In one embodiment, the mammal is a human.

### BRIEF DESCRIPTION OF THE DRAWINGS

FIG. 1 depicts the levels of circulating endothelial progenitor cells (CEPs) in a tumor after treatment with CA 1P, a Vascular Disrupting agent and/or AMD-3100, a CXCR4 antagonist agent. “Baseline” indicates CEP levels in an untreated tumor. “AMD+OXI” indicates tumors treated with a combination of AMD-3100 and CA1P. CEP levels were determined at 4 hours or 24 hours post-treatment.

FIG. 2 depicts the percentage of necrotic tissue “(% of Necrosis”) in MeWo tumors 3 days after treatment with CA1P and/or AMD-3100. “AMD+OXI” indicates tumors treated with a combination of AMD-3100 and CA1P. “Cont” indicates % necrosis in an untreated tumor. Necrosis area from total tumor area (n>20 fields/group) were calculated and plotted as the percentage of necrosis. *, 0.05>p>0.01; **, p<0.01.

FIG. 3 depicts the percentage of necrosis vessel perfusion and hypoxia in MeWo tumors treated with AMD-3100 and/or CA1P. “AMD+OXI” indicates tumors treated with a combination of AMD-3100 and CA1P. Summary of necrosis or perfusion and hypoxia area from total tumor area was calculated and plotted respectively. bars=100 μm; *, 0.05>p>0.01; **, p<0.01.

FIG. 4 depicts the tumor growth of MeWo human tumors grown in nude mice that were treated with CA1P, AMD-3100 or the combination of the two agents.

### DETAILED DESCRIPTION OF THE INVENTION

The invention is based, on the surprising and unexpected discovery that CXCR antagonists (and/or chemokine antagonists) can prevent the regrowth or relapse of tumor growth that may occur following treatment of a solid tumor with a VDA. In particular, the inventors have discovered that CXCR antagonists (or chemokine antagonists) can interfere with the recruitment and/or retention of bone-marrow-derived circulating endothelial progenitor cells (“CEPs”) within the tumor that occurs following treatment of the solid tumor with VDA therapy. CEPs have been shown to be a major determinant in tumor angiogenesis following VDA therapy (Shaked et al., Science, (2006), 313: 1785-1787). For example, VDAs can cause an rapid and pronounced increase (e.g., more that 3-4 fold) in the levels of CEPs in peripheral blood. These and other bone marrow derived proangiogenic cells home to the viable tumor rim which remains after VDA therapy and incorporate into the newly formed blood vessels, thereby contributing to tumor angiogenesis (e.g., by secreting growth factors such as vascular endothelial growth factor (VEGF)) and promoting the tumor re-growth that sometimes occurs following VDA treatment. The process whereby CEP and other bone marrow derived cells home to solid tumors is regulated in part by the secretion of the angiogenic chemokine factors (e.g., Stromal Cell Derived Factor 1 (SDF-1)) from solid tumors shortly following treatment with a VDA. These factors are thought to attract CXCR+ cells (e.g., CXCR4+ cells), and other cells which express cognate chemokine receptors on their cell surfaces, to the tumor site and promote their incorporation and retention in the solid tumor vasculature.

The inventors have made the surprising discovery that blocking the activity of proangiogenic chemokine receptors with a chemokine receptor antagonist can prevent the mobilization and retention of CEPs in a solid tumor (i.e., non-hematopoietic cancers) following VDA therapy and thereby enhance the efficacy of the VDA therapy. This result was highly unexpected since chemokine receptor antagonists have been known to augment (instead of inhibit) the mobilization of CEPs into peripheral blood (see, e.g., Shepherd et al., Blood, (2006), 108(12):3662-3667). Based on these previous findings, one skilled in the art would have expected that combination of a chemokine receptor antagonist (or chemokine antagonist) with a VDA would have further increased the mobilization of CEPs to a tumor, thereby reducing the efficacy of the VDA therapy. In contrast, the inventors have shown that combination therapy results in precisely the opposite therapeutic outcome.

Stromal Cell Derived Factor-1 (“SDF-1”, also known as CXCL12), the chemokine ligand of CXCR4, has been shown to stimulate the homing of CEPs and other hematopoietic progenitor cells into neo-angiogenic niches thereby supporting revascularization of ischemic tissue and tumor growth (reviewed in Petit et al., Trends in Immunol., 2007, 28 (7): 299-307). Accordingly, in alternative embodiments, the invention provides the administration of a VDA together with a chemokine antagonist (e.g., a SDF-1 antagonist) to potentiate the efficacy of a VDA. Without being bound to any particular theory, inhibition of biological function of SDF-1 (e.g., by blocking SDF-1 binding to CXCR4) will therefore inhibit the formation of the vasculature necessary for tumor regrowth following administration of the VDA. Any antagonist that interferes with the biological function of SDF-1 may be employed in the methods and compositions of the invention.

So that the invention can be more clearly understood, the following definitions are provided:

### I. Definitions

As used herein, the term “effective amount” of a compound or pharmaceutical composition refers to an amount sufficient to provide the desired anti-cancer effect or anti-tumor effect in an animal, preferably a human, suffering from cancer. Desired anti-tumor effects include, without limitation, the modulation of tumor growth (e.g. tumor growth delay), tumor size, or metastasis, the reduction of toxicity and side effects associated with a particular anti-cancer agent, the enhancement of tumor necrosis or hypoxia, the reduction of tumor angiogenesis, the reduction of tumor re-growth, reduced tumor retention of CEPs and other pro-angiogenic cells, the amelioration or minimization of the clinical impairment or symptoms of cancer, extending the survival of the subject beyond that which would otherwise be expected in the absence of such treatment, and the prevention of tumor growth in an animal lacking any tumor formation prior to administration, i.e., prophylactic administration.

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis, including tumor re-growth following treatment with an anticancer agent; reversing tumor growth and/or metastasis (including tumor shrinkage and/or eradication) and/or preventing tumor growth and/or metastasis.

“Synergistic effect”, as used herein refers to a greater-than-additive anti-cancer effect which is produced by a combination of two drugs, and which exceeds that which would otherwise result from individual administration of either drug alone. One measure of synergy between two drugs is the combination index (CI) method of Chou and Talalay (see Chang et al., Cancer Res. 45: 2434-2439, (1985)) which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. Another measurement of synergy is the fractional inhibitory concentration (FIC). This fractional value is determined by expressing the IC₅₀ of a drug acting in combination, as a function of the IC₅₀ of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

The term “anticancer agent” as used herein denotes a chemical compound or electromagnetic radiation (especially, X-rays) which is capable of modulating tumor growth or metastasis. When referring to use of such an agent with a combretastatin compound, the term refers to an agent other than a combretastatin compound. Unless otherwise indicated, this term can include one, or more than one, such agents. Thus, the term “anticancer agent” encompasses the use of one or more chemical compounds and/or electomagnetic radiation in the present methods and compositions. Where more than one anticancer agent is employed, the relative time for administration of the combretastatin compound can, as desired, be selected to provide a time-dependent effective tumor concentration of one, or more than one, of the anticancer agents.

As used herein, the term “combretastatin agent” or “combretastatin” denotes at least one member of the combretastatin family of compounds, derivatives or analogs thereof, their prodrugs (preferably phosphate prodrugs) and derivatives thereof, and salts of these compounds. Combretastatins include those anti-cancer compounds isolated from the South African tree Combretum caffrum, including without limitation, Combretastatins A-1, A-2, A-3, A-4, B-1, B-2, B-3, B-4, D-1, and D-2, and various prodrugs thereof, exemplified by Combretastatin A-4 phosphate (CA4P) compounds, Combretastatin A-1 diphosphate (CA1P) compounds and salts thereof (see for example Pettit et al, Can. J. Chem., (1982); Pettit et al., J. Org. Chem., 1985; Pettit et al., J. Nat. Prod., 1987; Lin et al., Biochemistry, (1989); Pettit et al., J. Med. Chem., 1995; Pettit et al., Anticancer Drug Design, (2000); Pettit et al., Anticancer Drug Design, 16(4-5): 185-93 (2001)). Other exemplary prodrugs of combrestatin agents include the cyclic phosph(oramid)ate prodrugs described in U.S. Pat. Nos. 7,205,404 and 7,303,739, which are incorporated by reference herein. Exemplary combretastatin derivatives retain cis-stilbene as a fundamental skeleton and exhibit tubulin polymerization inhibiting activity of 10 micromolar or less (e.g., 1 micromolar, 0.1 micromolar, 10 nanomolar, 1 nanomolar or less).

As used herein, the term combretastatin A-4 phosphate (“CA4P”) denotes as least one of combretastatin A-4 phosphate prodrugs, derivatives thereof, and salts of these compounds. As used herein, the term combretastatin A-1 diphosphate (“CA1P”) compound denotes as least one of combretastatin A-1 diphosphate prodrugs (e.g., OXi4503), derivatives thereof, and salts of these compounds.

As used herein, the term “prodrug” refers to a precursor form of the drug which is metabolically converted in vivo to produce the active drug. Thus, for example, combretastatin phosphate prodrug salts administered to an animal in accordance with the present invention undergo metabolic activation and regenerate combretastatin A-4 or combretastatin A-1 in vivo, e.g., following dissociation and exposure to endogenous non-specific phosphatases in the body. Preferred prodrugs of the present invention include the phosphate, phosphate ester, phosphoramidate, phosphoramidate ester, or amino acid acyl groups as defined herein. Exemplary phosphate esters include —OP(O)(O-alkyl)₂ or salts of the phosphate group, for example —OP(O)(O⁻NH₄⁺)₂. In preferred embodiments, a prodrug of the invention comprises a substitution of a phenolic moiety or amine moiety of the active drug with a phosphate, phosphoramidate, or amino acid acyl group. A wide variety of methods for the preparation of prodrugs are known to those skilled in the art (see, for example, Pettit and Lippert, Anti-Cancer Drug Design, (2000), 15, 203-216).

As explained above, the present invention is directed towards a pharmaceutical composition that modulates growth or metastasis of tumors, particularly solid tumors, using a pharmaceutical composition of the present invention, along with methods of modulating tumor growth or metastasis, for example, with a pharmaceutical composition of the present invention.

The term “subject” is intended to include mammals suffering from or afflicted with a tumor. Exemplary subjects include humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a cancer.

As used herein, the terms “tumor”, “tumor growth” or “tumor tissue” can be used interchangeably, and refer to an abnormal growth of tissue resulting from uncontrolled progressive multiplication of cells and serving no physiological function.

In particularly preferred embodiments, the methods of the invention are used to treat solid tumors. As is well-known in the art, solid tumors are quite distinct from non-solid tumors, such as those found in hemopoietic-related cancers. A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastric cancer, pancreatic cancer, breast cancer, ovarian cancer, fallopian tube cancer, primary carcinoma of the peritoneum, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In other embodiments, the methods of the invention are used to treat non-solid tumors. Examples of non-solid tumors include leukemias, such as myeloid leukemias and lymphoid leukemias, myelomas, and lymphomas. Particular forms of non-solid tumors include acute myelitic leukemia (AML), acute lymphatic leukemia (ALL), multiple myeloma (MM), chronic myelogenous leukemia (CML), hairy cell leukemia (HCL), acute promyelocytic leukemia (APL), and chronic lymphocytic leukemia (CLL). In a particularly preferred embodiment, the methods of the invention are used to treat chronic myelomonocytic leukemia (CMML).

In other embodiments, tumors comprising dysproliferative changes (such as metaplasias and dysplasias) can be treated or prevented with a pharmaceutical composition or method of the present invention in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68 to 79). Hyperplasia is a form of controlled cell proliferation involving an increase in cell number in a tissue or organ, without significant alteration in structure or function. For example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J.B. Lippincott Co., Philadelphia.

Other examples of tumors that are benign and can be treated or prevented in accordance with a method of the present invention include arteriovenous (AV) malformations, particularly in intracranial sites and myelomas.

The term “time-dependent effective tumor concentration,” as used herein, denotes a concentration of the other anticancer agent in the tumor tissue over time (i.e, from administration until the agent is cleared from the body) which potentiates the action of the combination of the combretastatin compound and other anticancer agent.

The phrase “Peak Tumor Concentration Agents” refers to anticancer agents which are most efficacious at high tumor concentrations yet are rapidly cleared from the tumor tissue. Such agents may be administered simultaneously with or in close temporal proximity to (e.g., as is clinically feasible, especially within one hour of) the administration of the combretastatin compound in accordance with the invention. Exemplary Peak Tumor Concentration Agents include, without limitation, alkylating agents (e.g. cytoxan and mitomycin C) and metal coordination complexes such as cisplatin, oxaliplatin and carboplatin.

The phrase “Duration Exposure Agents” as used herein refers to agents which can be effective at relatively low tumor concentrations yet which require certain tumor tissue exposure times to be most effective. Such agents may be administered sequentially in any order with a combretastatin compound in accordance with the invention, provided that a sufficient delay is allowed between administrations to potentiate the combination. In one embodiment of the method of the invention, the Duration Exposure Agent is administered after the administration of the combretastatin compound. Exemplary Duration Exposure Agents include, without limitation, taxanes such as paclitaxel and docetaxel, etoposide, etoposide phosphate, immunotoxins, and epothilones.

The phrase “High AUC Agents” as used herein refers to those agents which show greatest efficacy when present at high concentrations in tumor tissue for extended time periods. Such agents are may be administered sequentially with a combretastatin compound in accordance with the invention, wherein the High AUC Agent is administered first, followed by the combretastatin compound, provided that a sufficient delay is allowed between administrations to potentiate the combination. Exemplary High AUC Agents include, without limitation, adriamycin, CPT-11 (irinotecan), and topotecan.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl, and isobutyl and sec-butyl. Moreover, the term C₃₋₇-cycloalkyl includes, but is not limited to, cyclopropyl, cyclopentyl, cyclohexyl and cycloheptyl. As discussed below, these alkyl groups, as well as cycloalkyl groups, may be further substituted.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons. Likewise, preferred cycloalkyls have from 4-7 carbon atoms in their ring structure, and more preferably have 5 or 6 carbons in the ring structure.

Moreover, alkyl (e.g., methyl, ethyl, propyl, butyl, pentyl, hexyl, etc.) includes both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted” is intended to describe moieties having substituents replacing a hydrogen on one or more atoms, e.g. C, O or N, of a molecule. Such substitutents can include electron-withdrawing groups or electron-withdrawing atoms. Such substituents can include, for example, oxo, alkyl, alkoxy, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, morpholino, phenol, benzyl, phenyl, piperizine, cyclopentane, cyclohexane, pyridine, 5H-tetrazole, triazole, piperidine, or an aromatic or heteroaromatic moiety, and any combination thereof.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, thiol, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety, and any combination thereof. In certain embodiments, a carbonyl moiety (C═O) can be further derivatized with an oxime moiety, e.g., an aldehyde moiety can be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “amine” or “amino” should be understood as being broadly applied to both a molecule, or a moiety or functional group, as generally understood in the art, and can be primary, secondary, or tertiary. The term “amine” or “amino” includes compounds where a nitrogen atom is covalently bonded to at least one carbon, hydrogen or heteroatom. The terms include, for example, but are not limited to, “alkyl amino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

In a particular embodiment of the invention, the term “amine” or “amino” refers to substituents of the formulas N(R⁸)R⁹ or C₁₋₆—N(R⁸)R⁹, wherein R⁸ and R⁹ are each, independently, selected from the group consisting of —H and —(C₁₋₆alkyl)₀₋₁G, wherein G is selected from the group consisting of —COOH, —H, —PO₃H, —SO₃H, —Br, —Cl, —F, —O—C₁₋₄alkyl, —S—C₁₋₄alkyl, aryl, —C(O)OC₁-C₆-alkyl, —C(O)C₁₋₄alkyl-COOH, —C(O)C₁-C₄-alkyl, —C(O)-aryl, substituted or unsubstituted aryl, substituted or unsubstituted heteroaryl, substituted or unsubstituted cycloalkyl; or N(R⁸)R⁹ is pyrrolyl, tetrazolyl, pyrrolidinyl, pyrrolidinyl-2-one, dimethylpyrrolyl, imidazolyl and morpholino.

The term “aryl” includes groups, including 5- and 6-membered single-ring aromatic groups that can include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure can also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrahydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “electron-withdrawing group” “or electron-withdrawing atom” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (Σ) constant. This well known constant is described in many references, for instance, J. March, Advanced Organic Chemistry, McGraw Hill Book Company, New York, (1977 edition) pp. 251-259. The Hammett constant values are generally negative for electron donating groups (Σ[P]=−0.66 for NH₂) and positive for electron withdrawing groups (Σ[P]=0.78 for a nitro group), wherein Σ[P] indicates para substitution. Non-limiting examples of electron-withdrawing groups include nitro, acyl, formyl, sulfonyl, trifluoromethyl, cyano, chloride, carbonyl, thiocarbonyl, ester, imino, amido, carboxylic acid, sulfonic acid, sulfinic acid, sulfamic acid, phosphonic acid, boronic acid, sulfate ester, hydroxyl, mercapto, cyano, cyanate, thiocyanate, isocyanate, isothiocyanate, carbonate, nitrate and nitro groups and the like. Exemplary electron-withdrawing atoms include, but are not limited to, an oxygen atom, a nitrogen atom, a sulfur atom or a halogen atom, such as a fluorine, chlorine, bromine or iodine atom. It is to be understood that, unless otherwise indicated, reference herein to an acidic functional group also encompasses salts of that functional group in combination with a suitable cation. Non-limiting examples of electron donating groups include, but are not limited to, a primary amino, secondary amino, tertiary amino, hydroxy, alkoxy, aryloxy, alkyl, or combinations thereof.

The description of the disclosure herein should be construed in congruity with the laws and principals of chemical bonding. For example, it may be necessary to remove a hydrogen atom in order accommodate a substitutent at any given location. Furthermore, it is to be understood that definitions of the variables (i.e., “R groups”), as well as the bond locations of the generic formulae of the invention, will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like. Other pharmaceutically acceptable salts include alkali metal cations such as Na, K, Li; alkali earth metal salts such as Mg or Ca; or organic amine salts such as those disclosed in PCT International Application Nos. WO02/22626 or WO00/48606 and U.S. Pat. Nos. 6,855,702 and 6,670,344, which are incorporated herein by reference in their entireties. Particularly preferred salts include organic amine salts such tromethamine (TRIS) and amino acid salts such as histidine. Other exemplary salts which can be synthesized using the methods of the invention include those described in U.S. Pat. No. 7,018,987, which is incorporated by reference herein.

### II. Vascular Disrupting Agents (VDAs)

Vascular Disrupting Agents (“VDAs”), also known as vascular damaging agents or vascular targeting agents, are a separate class of antivascular chemotherapeutics. In contrast to anti-angiogenic drugs, which disrupt the new microvessel formation of developing tumors, VDAs attack solid tumors by selectively targeting the established tumor vasculature and causing extensive shutdown of tumor blood flow. A single dose of a VDA can cause a rapid and selective shutdown of the tumor neovasculature within a period of minutes to hours, leading eventually to tumor necrosis by induction of hypoxia and nutrient depletion. This vascular-mediated cytotoxic mechanism of VDA action is quite divorced from that of anti-angiogenic agents, which inhibit the formation of new tumor vascularization rather than interfering with the existing tumor vasculature. Other agents have been known to disrupt tumor vasculature, but differ in that they also manifest substantial normal tissue toxicity at their maximum tolerated dose. In contrast, genuine VDAs retain their vascular shutdown activity at a fraction of their maximum tolerated dose. It is thought that tubulin-binding VDAs selectively destabilize the microtubule cytoskeleton of tumor endothelial cells, causing a profound alteration in the shape of the cell which ultimately leads to occlusion of the tumor blood vessel and shutdown of blood flow to the tumor (Kanthou et al., Blood, 2002; Cooney et al., Curr Oncol Rep. 2005 7(2):90-5; Chaplin et al., Curr Opin Investig Drugs, (2006), 7(6):522-8).

A particularly promising subclass of VDAs are the combretastatins. Derived from the South African tree Combretum caffrum, combretastatins such as combretastatin A-4 (CA-4) were initially identified in the 1980's as potent inhibitors of tubulin polymerization. CA-4, and other combretastatins (e.g. combretastatin A-1 (CA-1)) have been shown to bind a site at or near the colchicine binding site on tubulin with high affinity. In vitro studies clearly demonstrated that combretastatins are potent cytotoxic agents against a diverse spectrum of tumor cell types in culture. CA4P and CA1P, respective phosphate prodrugs of CA-4 and CA-1, were subsequently developed to combat problems with aqueous insolubility (see U.S. Pat. Nos. 4,996,237; 5,409,953; and 5,569,786, each of which is incorporated herein by reference). Surprisingly, CA1P and CA4P have also been shown to cause a rapid and acute shutdown of the blood flow to tumor tissue that is separate and distinct from the anti-proliferative effects of the agents on tumor cells themselves. A number of studies have shown that combretastatins cause extensive shut-down of blood flow within the tumor microvasculature, leading to secondary tumor cell death (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). Blood flow to normal tissues is generally far less affected by CA4P and CA1P than blood flow to tumors, although blood flow to some organs, such as spleen, skin, skeletal muscle and brain, can be inhibited (Tozer et al., Cancer Res., 59: 1626-34 (1999)).

In light of the novel, non-cytotoxic, mode of action of combretastatins, there is considerable interest in exploiting the novel “vascular targeting” of these agents for cancer treatment. Single agent efficacy has been reported for CA4P using a frequent dosing regimen. Another report suggested that large tumors can, in some cases, be more responsive to CA4P therapy than small tumors. However, many tumors harvested from animals treated with CA4P reveal central necrosis surrounded by a rim of viable cells (Dark et al., Cancer Res., 57: 1829-34, (1997); Chaplin et al., Anticancer Res., 19: 189-96, (1999)). This rim of surviving cells is most likely a consequence of the shared normal vessel circulation between the perimeter of tumours and neighbouring normal tissue. The addition of a CXCR antagonist (or chemokine antagonist) according to the present invention inhibits tumor regrowth from this rim of viable cells.

Exemplary combretastatin salts contemplated for use in the methods of the invention are described in WO 99/35150; WO 01/81355; U.S. Pat. Nos. 6,670,344; 6,538,038; 5,569,786; 5,561,122; 5,409,953; 4,996,237 which are incorporated herein by reference in their entirety.

Exemplary combretastatin derivatives or analogs of combretastatins are described in Singh et al., J. Org. Chem., 1989; Cushman et al, J. Med. Chem., 1991; Getahun et al, J. Med. Chem., 1992; Andres et al, Bioorg. Med. Chem. Lett., 1993; Mannila, et al., Liebigs. Ann. Chem., 1993; Shirai et al., Bioorg. Med. Chem. Lett., 1994; Medarde et al., Bioorg. Med. Chem. Lett., 1995; Wood et al, Br. J. Cancer, 1995; Bedford et al., Bioorg. Med. Chem. Lett., 1996; Dorr et al., Invest. New Drugs, 1996; Jonnalagadda et al., Bioorg. Med. Chem. Lett., 1996; Shirai et al., Heterocycles, 1997; Aleksandrzak, et al., Anticancer Drugs, 1998; Chen et al., Biochem. Pharmacol., 1998; Ducki et al., Bioorg. Med. Chem. Lett., 1998; Hatanaka et al., Bioorg Med. Chem. Lett., 1998; Medarde et al., Eur. J. Med. Chem., 1998; Medina et al., Bioorg. Med. Chem. Lett., 1998; Ohsumi et al., Bioorg. Med. Chem. Lett., 1998; Ohsumi et al., J. Med. Chem., 1998; Pettit, et al., J. Med. Chem., 1998; Shirai et al., Bioorg Med. Chem. Lett., 1998; Banwell et al., Aust. J. Chem., 1999; Medarde et al., Bioorg. Med. Chem. Lett., 1999; Shan et al., PNAS, 1999; Combeau et al., Mol. Pharmacol., 2000; Pettit et al., J. Med. Chem., 2000; Pinney et al., Bioorg. Med. Chem. Lett., 2000; Flynn et al., Bioorg. Med. Chem. Lett., 2001; Gwaltney et al., Bioorg Med. Chem. Lett., 2001; Lawrence et al., 2001; Nguyen-Hai et al., Bioorg. Med. Chem. Lett., 2001; Xia et al., J. Med. Chem., 2001; Tahir et al., Cancer Res., 2001; Wu-Wong et al., Cancer Res., 2001; Janik et al, Bioorg. Med. Chem. Lett., 2002; Kim et al., Bioorg Med Chem. Lett., 2002; Li et al., Bioorg. Med. Chem. Lett., 2002; Nam et al., Bioorg Med. Chem. Lett., 2002; Wang et al., J. Med. Chem. 2002; Hsieh et al., Bioorg. Med. Chem. Lett., 2003; Hadimani et al., Bioorg. Med. Chem. Lett., 2003; Mu et al., J. Med. Chem, 2003; Nam et al., Curr. Med. Chem., 2003; Pettit et al, J. Med. Chem., 2003; Gaukroger et al., Org Biomol Chem. 2003; Bailly et al., J Med Chem. 2003; Sun et al., Anticancer Res. 2004; Sun et al., Bioorg Med Chem Lett. 2004; Liou et al., J Med Chem. 2004; Perez-Melero et al., Bioorg Med Chem Lett. 2004; Liou et al., J Med Chem. 2004; Mamane et al., Chemistry. 2004; De Martini et al, J Med Chem. 2004; Ducki et al, J Med Chem. 2005; Maya et al., J Med Chem. 2005; Medarde et al., J Enzyme Inhib Med Chem. 2004; Simoni et al, J Med Chem. 2005; Sanchez et al., Bioorg Med Chem. 2005; Vongvanich et al., Planta Med. 2005; Tron et al., J Med Chem. 2005; Borrel et al., Bioorg Med Chem. 2005; Hsieh et al., Curr Pharm Des. 2005; Lawrence et al, Curr Pharm Des. 2005; Hadfield et al., Eur J Med Chem. 2005; Pettit et al., J Med Chem. 2005; Coggioloa et al., Bioorg Med Chem Lett. 2005; Kaffy et al., Org Biomol Chem. 2005; Mateo et al, J Org Chem. 2005; LeBlanc et al., Bioorg Med Chem. 2005; Srivistava et al., Bioorg Med Chem. 2005; Nguyen et al., J Med Chem. 2005; Kong et al., Chem Biol. 2005; Li et al, Bioorg Med Chem Lett. 2005; Pettit et al, J Nat Prod. 2005; Nicholson et al, Anticancer Drugs. 2006; Monk et al., Bioorg Med Chem. 2006; De Martino et al., J Med Chem. 2006; Peifer et al., J Med Chem. 2006; Kaffy et al., Bioorg Med Chem. 2006; Banwell et al., Bioorg Med Chem. 2006; Dupeyre et al., Bioorg Med Chem. 2006 Simoni et al, J Med Chem. 2006; Tron et al., J Med Chem. 2006; Romagnoli et al, J Med Chem. 2006; Pandit et al., Bioorg Med Chem. 2006; Nakamura et al., Chem Med Chem. 2006; Pirali et al., J Med Chem. 2006; Bellina et al., Bioorg Med Chem Lett. 2006; Hu et al, J Med Chem. 2006; Chang et al., J Med Chem. 2006; Thomson et al., Mol Cancer Ther. 2006; Fortin et al., Bioorg Med Chem Lett., 2007; Duan et al., J Med Chem., 2007; Zhang et al., J Med Chem. 2007; Wu et al., Bioorg Med Chem Lett. 2007; Sun et al., Bioorg Med Chem Lett. 2007, WO 07/140,662; WO 07/059,118; WO 06/138427; WO 06/036743; WO 05/007635, WO 03/040077, WO 03/035008, WO 02/50007, WO 02/14329; WO 01/12579, WO 01/09103, WO 01/81288, WO 01/84929, WO 00/48590, WO 00/73264, WO 00/06556, WO 00/35865, WO 99/34788, WO 99/48495, WO 92/16486, U.S. Pat. Nos. 7,312,241; 7,223,747; 7,220,784; 7,135,502; 7,125,906; 7,105,695; 7,105,501; 7,087,627; 7,030,123; 7,078,552; 7,030,123; 7,018,987; 6,992,106; 6,919,324; 6,846,192, 6,855,702; 6,849,656; 6,794,384; 6,787,672, 6,777,578, 6,723,858, 6,720,323, 6,433,012, 6,423,753, 6,201,001, 6,150,407, 6,169,104, 5,731,353, 5,674,906, 5,430,062, 5,525,632, 4,996,237 and 4,940,726, each of which are incorporated herein by reference in their entirety.

In one exemplary embodiment, a combretastatin derivate is the amine or serinamide derivative of CA4, e.g. AVE8032 (Aventis Pharma, France). In another exemplary embodiment, a combretastatin derivative is ZD6126 (AstraZeneca, UK).

In particular embodiments, a combretastatin derivative is a compound of Formula V:

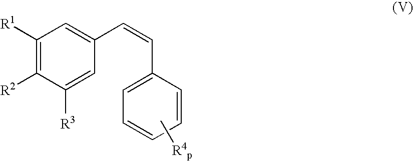

wherein
each of R¹, R² and R³, independently of the others, is selected from the group consisting of hydrogen, C₁₋₆ alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR⁷ substituted with one or more of the same or R⁷ or R⁶, —B(OR⁷)₂, —B(NR⁸R⁸)₂, —(CH₂)ₘ—R⁶, —(CHR⁷)ₘ—R⁶, —O—(CH₂)ₘ—R⁶, —S—(CH₂)ₘ—R⁶, —O—CHR⁷R⁶, —O—CR⁷(R⁶)₂, O—(CHR⁷)ₘ—R⁶, —O—(CH₂)ₘ—CH[(CH₂)ₘR⁶]R⁶, —S—(CHR⁷)ₘ—R⁶, —C(O)NH—(CH₂)ₘ—R⁶, —C(O)NH—(CHR⁷)ₘ—R⁶, —O—(CH₂)ₘ—C(O)NH—(CH₂)ₘ—R⁶, —S—(CH₂)ₘ—C(O)NH—(CH₂)ₘ—R⁶, —O—(CHR⁷)ₘ—C(O)NH—(CHR⁷)ₘ—R⁶, —S—(CHR⁷)ₘ—C(O)NH—(CHR⁷)ₘ—R⁶, —NH—(CH₂)ₘ—R⁶, —NH—(CHR⁷)ₘR⁶, —NH[(CH₂)ₘR⁶], —N[(CH₂)ₘR⁶]₂, —NH—C(O)—NH—(CH₂)ₘ—R⁶, —NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate
each R⁶ is a suitable group independently selected from the group consisting of ═O, —OR⁷, C₁₋₃ haloalkyloxy, —OCF₃, ═S, —SR⁷, ═NR⁷, ═NOR⁷, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(OH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NHC(O)]ₙOR⁷, —[NR⁷C(O)]ₙOR⁷, —[NHC(O)]ₙNR⁸R⁸, —[NR⁷C(O)]ₙNR⁸R⁸, —[NHC(NH)]ₙNR⁸R⁸ and —[NR⁷C(NR⁷)]ₙNR⁸R⁸;
each R⁷ is independently selected from the group consisting of hydrogen, C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R⁷ or suitable R⁶ groups; each m independently is an integer from 1 to 3;
each n independently is an integer from 0 to 3;
p is an integer from 1 to 5, and

wherein two adjacent R⁴ groups and their intervening atoms are bonded to form a 5-8 membered ring fused to the central phenyl group.

In a particularly preferred embodiment, the combretastatin agent is a compound of Formula II:

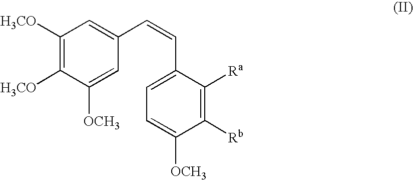

or a pharmaceutically acceptable salt thereof wherein R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and

R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

In a preferred embodiment R^{a} is a phosphate of formula:

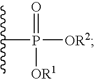

and R^{b} is a phosphate of formula:

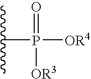

wherein OR¹, OR², OR³ and OR⁴ are each, independently, H, —O⁻QH⁺ or —O⁻M⁺, wherein M⁺ is a monovalent or divalent metal cation, and Q is, independently:

a) an amino acid containing at least two nitrogen atoms where one of the nitrogen atoms, together with a proton, forms a quaternary ammonium cation QH⁺; or

b) an organic amine containing at least one nitrogen atom which, together with a proton, forms a quaternary ammonium cation, QH⁺.

In a particular embodiment, the combrestatin agent is a compound of the Formula IIb:

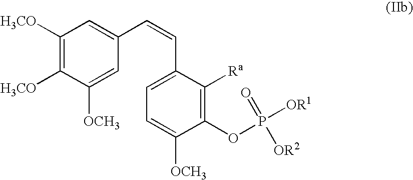

wherein

R^{a} is H or OP(O)(OR³)OR⁴; and

OR¹, OR², OR³ and OR⁴ are each, independently, H, —O⁻QH⁺ or —O⁻M⁺, wherein M⁺ is a monovalent or divalent metal cation, and Q is, independently:

a) an amino acid containing at least two nitrogen atoms where one of the nitrogen atoms, together with a proton, forms a quaternary ammonium cation QH⁺; or

b) an organic amine containing at least one nitrogen atom which, together with a proton, forms a quaternary ammonium cation, QH⁺.

In one embodiment of Formula IIb, R^{a} is H, one of OR¹ and OR² is hydroxyl, and the other is —O⁻QH⁺ where Q is L-histidine. In another embodiment of Formula IIb, R^{a} is H, one of OR¹ and OR² is hydroxyl and the other is —O⁻QH⁺ and Q is tris(hydroxymethyl)amino methane (“TRIS”).

In another embodiment of Formula IIb, R^{a} is H or OP(O)(OR³)OR⁴, and R¹, R², R³ and R⁴ are each, independently, an aliphatic organic amine, alkali metals, transition metals, heteroarylene, heterocyclyl, nucleoside, nucleotide, alkaloid, amino sugar, amino nitrile, or nitrogenous antibiotic.

In another embodiment of Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

In another embodiment, Formula IIb is represented by a compound of Formula III:

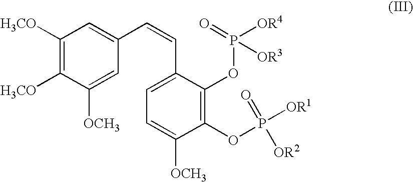

wherein OR¹, OR², OR³ and OR⁴ are each, independently, H, —O⁻QH⁺ or —O⁻M⁺, wherein M⁺ is a monovalent or divalent metal cation, and Q is, independently:

a) an amino acid containing at least two nitrogen atoms where one of the nitrogen atoms, together with a proton, forms a quaternary ammonium cation QH⁺; or

b) an organic containing at least one nitrogen atom which, together with a proton, forms a quaternary ammonium cation, QH⁺.

In one embodiment of Formula III, at least one of OR¹, OR², OR³ and OR⁴ is hydroxyl, and at least one of OR¹, OR², OR³ and OR⁴ is —O⁻QH⁺, where Q is L-histidine. In another embodiment of Formula III, at least one of OR¹, OR², OR³ and OR⁴ is hydroxyl, and at least one of OR¹, OR², OR³ and OR⁴ is TRIS.

In another aspect, the invention provides a pharmaceutical composition comprising a compound of Formula I and a compound of Formula IV:

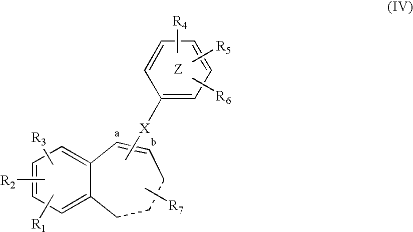

wherein

the dashed lines independently indicate a single or double bond;

X is selected from the group consisting of a single bond, CH₂, O, S, N(H), and C(O);

R₁, R₂, R₃, R₄, R₅, R₆ and R₇ are each, independently, selected from the group consisting of H, halogen, lower alkyl, lower alkoxy, hydroxyl, amine, phosphate, phosphoramidate, and amino acid acyl group; and

phenyl ring “Z” is bonded to either carbon “a” or “b.”

In one embodiment, the compound of Formula IV is selected from the group consisting of (1) 3-Methoxy-8-(3,4,5-trimethoxy-phenyl)-6,7-dihydro-5H-benzocycloheptenel; (2) 3-Methoxy-9-(3,4,5-trimethoxy-phenyl)-6,7-dihydro-5H-benzocycloheptene; (3) 2-Methoxy-6-(3,4,5-trimethoxy-phenyl)-8,9-dihydro-7H-benzocyclohepten-1-ol; (4) 2-Methoxy-5-(3,4,5-trimethoxy-phenyl)-8,9-dihydro-7H-benzocyclohepten-1-ol; (5) 2-Methoxy-6-(3,4,5-trimethoxy-phenyl)-8,9-dihydro-7H-benzocyclohepten-1-ol; (6) 2-Methoxy-5-(3,4,5-trimethoxy-phenyl)-7,8,9,10-tetrahydro-benzocycloocten-1-ol; (9) 3-Methoxy-6-(1,2,3-trimethoxy-8,9-dihydro-7H-benzocyclohepten-5-yl)-benzene-1,2-diol; (11) 3-Methoxy-6-(1,2,3-trimethoxy-8,9-dihydro-7H-benzocyclohepten-5-yl)-benzene-1,2-diol; (12) 2-Methoxy-5-(1,2,3-trimethoxy-8,9-dihydro-7H-benzocyclohepten-5-yl)-phenol; (13) 3-Methoxy-6-(1,2,3-trimethoxy-7,8,9,10-tetrahydro-benzocycloocten-5-yl)-benzene-1,2-diol; (14) 2-Methoxy-5-(1,2,3-trimethoxy-7,8,9,10-tetrahydro-benzocycloocten-5-yl)-phenol; (31) 2-Methoxy-5-(1,2,3-trimethoxy-8,9-dihydro-7H-benzocyclohepten-6-yl)-phenol; (32) 3-Methoxy-6-(1,2,3-trimethoxy-8,9-dihydro-7H-benzocyclohepten-6-yl)-benzene-1,2-diol; (15) (1-Hydroxy-2-methoxy-8,9-dihydro-7H-benzocyclohepten-6-yl)-(3,4,5-trimethoxy-phenyl)-methanone; (16) (1-Hydroxy-2-methoxy-8,9-dihydro-7H-benzocyclohepten-5-yl)-(3,4,5-trimethoxy-phenyl)-methanone; (17) (1-Hydroxy-2-methoxy-7,8,9,10-tetrahydro-benzocycloocten-6-yl)-(3,4,5-trimethoxy-phenyl)-methanone; (18) (1-Hydroxy-2-methoxy-7,8,9,10-tetrahydro-benzocycloocten-5-yl)-(3,4,5-trimethoxy-phenyl)-methanone; (23) (2,3-Dihydroxy-4-methoxy-phenyl)-(1,2,3-trimethoxy-8,9-dihydro-7H-benzocyclohepten-5-yl)-methanone; (24) (3-Hydroxy-4-methoxy-phenyl)-(1,2,3-trimethoxy-8,9-dihydro-7H-benzocyclohepten-5-yl)-methanone; (25) (2,3-Dihydroxy-4-methoxy-phenyl)-(1,2,3-trimethoxy-7,8,9,10-tetrahydro-benzocycloocten-5-yl)-methanone and (26) (3-Hydroxy-4-methoxy-phenyl)-(1,2,3-trimethoxy-7,8,9,10-tetrahydro-benzocycloocten-5-yl)-methanone. Additional compounds of Formula IV are disclosed in International PCT publication No. WO 2006/138427A2, published Dec. 28, 2006, which is incorporated by reference herein in its entirety.

### III. Chemokine Receptor Antagonists

In certain aspects of the invention, a VDA is administered together with a chemokine receptor antagonist. Chemokines are chemotactic proteins which stimulate the migration of blood cells towards a site of infection or a tumor. Chemokines bind chemokine receptors on the surface of these blood cells, thereby stimulating homing and migration of the blood cells to the source the chemokine (for reviews, see Well T N et al., Immunol. Lett., 1999, 65: 35-40; Zlotnik A et al., Crit. Rev. Immunol., (1999), 19: 1-47; Rossi and Zlotnik, Annu. Rev. Immunol., (2000), 18: 217; Zlotnik et al., Immunity, (2000), 12:121). A number of chemokine receptors are preferentially expressed on leukocytes, including Th1-specific chemokine receptors such as CXCR3 and CCR5 and Th2-specific chemokine receptors such as CCR4, CCR8, and CXCR4. Any antagonist which interferes with the biological function of a chemokine receptor may employed in the methods of the invention. Art-recognized antagonists include antibodies which bind to chemokine receptors and interference with ligand binding, siRNAs which bind and cleave chemokine receptor mRNAs and prevent their expression, and the like.

In certain exemplary embodiments, a chemokine receptor antagonist employed in the methods of the invention is a CXCR4 antagonist. CXCR4, like many chemokine receptors is a Gi-coupled receptor that is specific for SDF-1 (also known as CXCL12, see, Bleul et al., Nature, 1996, 382: 829).

In certain embodiments, an CXCR4 antagonist employed in the methods of the invention is a compound of the Formula Ia:

Z-linker-Y (Ia)

wherein Z and Y are each, independently, a cyclic polyamine moiety having a total of 9 to 32 atoms and from 2 to 8 optionally substituted nitrogens spaced by two or more optionally substituted carbon atoms from each other, and which may optionally comprise additional heteroatoms besides nitrogen and/or may be fused to an additional ring system. As used herein, “linker” represents a bond, alkylene, or may comprise aryl, fused aryl, oxygen atoms contained in an alkylene chain, or may contain keto groups or nitrogen or sulfur atoms.

In certain preferred embodiments, an CXCR4 antagonist employed in the methods of the invention is a compound of the Formula I:

Z-R—Ar—R′—Y (I)

wherein Z and Y are each, independently, a cyclic polyamine moiety having a total of 9 to 24 atoms and from 2 to 6 optionally substituted nitrogens spaced by two or more optionally substituted carbon atoms from each other, and which may optionally comprise a fused aromatic or heteroaromatic ring;

R and R′ are each, independently, selected from the group consisting of straight, branched, or cyclic C₁₋₆-alkyl groups; and

Ar is an aromatic or heteroaromatic ring, optionally substituted at single or multiple positions with electron-donating or electron-withdrawing groups;

and pharmaceutically acceptable acid addition salts and metal complexes thereof.

In one embodiment of Formula I or Ia, Z and Y are each, independently, a cyclic polyamine moiety having a total of 14 to 20 atoms and from 3 to 6 optionally substituted amino nitrogens spaced by two or more optionally substituted carbon atoms from each other. In another embodiment of Formula I, Ar is phenyl. In still another embodiment of Formula I, R and R′ are CH₂.

In preferred embodiments, Formula I is represented by 1,1′-[1,3-phenylenebis(methylene)]-bis-1,4,8,11-tetra-azacyclotetradecane; 1,1′-[1,4-phenylenebis(methylene)]-bis-1,4,8,11-tetra-azacyclotetradecane; 1,1′-[3,3′-biphenylene-bis-(methylene)]-bis-1,4,8,11-tetraazacyclotetradecane; 11,11′-[1,4-phenylene-bis-(methylene)]-bis-1,4,7,11-tetraazacyclotetradecane; 1,11′-[1,4-phenylene-bis-(methylene)]-1,4,8,11-tetraazacyclotetradecane-1,4,7,11-tetraazacyclotetradecane; 1,1′-[2,6-pyridine-bis-(methylene)]-bis-1,4,8,11-tetraazacyclotetradecane; 1,1-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[5-nitro-1,3-phenylenebis(methylene)]bis-1,4,8,11-tetraazacyclotetradecane; 1,1′-[2,4,5,6-tetrachloro-1,3-phenyleneis(methylene)]bis-1,4,8,11-tetraazacyclotetradecane; 1,1′-[2,3,5,6-tetra-fluoro-1,4-phenylenebis(methylene)]bis-1,4,8,11-tetraazacyclotetradecane; 1,1′-[1,4-naphthylene-bis-(methylene)]bis-1,4,8,11-tetraazacyclotetradecane; 1,1′-[1,3-phenylenebis-(methylene)]bis-1,5,9-triazacyclododecane; 1,1′-[1,4-phenylene-bis-(methylene)]-1,5,9-triazacyclododecane; 1,1′-[3,3′-biphenylene-bis-(methylene)]-bis-1,4,8,11-tetraazacyclotetradecane; 1,1′-[2,6-pyridine-bis-(methylene)]-bis-1,4,8,11-tetraazacyclotetradecane; 1,1′-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[2,5-dimethyl-1,4-phenylenebis-(methylene)]-bis-1,4,8,11-tetraazacyclotetradecane; 1,1′-[2,5-dichloro-1,4-phenylenebis-(methylene)]-bis-1,4,8,11-tetraazacyclotetradecane; 1,1′-[2-bromo-1,4-phenylenebis-(methylene)]-bis-1,4,8,11-tetraazacyclotetradecane; or 1,1′-[6-phenyl-2,4-pyridinebis-(methylene)]-bis-1,4,8,11-tetraazacyclotetradecane; and pharmaceutically acceptable salts thereof.

In another embodiment, a CXCR4 antagonist of the invention is a cyclic polyamine having from 9-24 carbon and including 3-5 nitrogen atoms, for example, 1,5,9,13-tetraazacyclohexadecane; 1,5,8,11,14-pentazacyclohexadecane; 1,4,8,11-tetraazacyclohexadecane; 1,5,9-triazacyclododecane; and 1,4,7,10-tetraazacyclododecane. Other embodiments are 3,7,11,17-tetraazabicyclo(13.3.1)heptadeca-1(17),13,15-triene; 4,7,10,17-tetraazabicyclo(13.3.1)heptadeca-1(17),13,15-triene; 1,4,7,10-tetraazacyclotetradecane; 1,4,7-triazacyclotetradecane; and 4,7,10-triazabicyclo(13.3.1)heptadeca-1(17),13,15-triene.

In preferred embodiments, the CXCR4 antagonist employed in the methods of the invention is selected from the group consisting of AMD 3465, AMD 7049, AMD 7049, AMD 7050, AMD 7051, AMD 7058, AMD 7059, AMD 7063, AMD 3538, AMD 3500, AMD 3499, AMD 3498, AMD 3497, AMD 3516, AMD 3530, AMD 3517, AMD 3544, AMD 3543, AMD 3529, AMD 7049, AMD 7050, AMD 7051, AMD 7059, AMD 7063, AMD 7058, AMD 7032, AMD 7048, AMD 7060, AMD 7061, AMD 3451, AMD 3454, AMD 3472, AMD 3526, AMD 3100, AMD 3484, AMD 3100, AMD 8630, AMD 7097, AMD 8631, AMD 7450, AMD 7463, and AMD11070. These compounds, and other CXCR4 antagonists that can be used in the pharmaceutical compositions of the invention, are described in International Patent Application Publication Nos. WO 00/02870 and WO 03/55876, U.S. Pat. No. 5,583,131, Lukacs et al., Am. J. Pathology. 2002, 160: 1353-1360; and Matthys et. al, J Immunol. 2001, 167(8):4686-92 all of which are incorporated herein by reference in their entirety.

A particularly preferred CXCR4 antagonist is the symmetric bicyclam AMD3100 (1,1′-[1,4-phenylenebis(methylene)]-bis-1,4,8,11-tetra-azacyclotetradecane (MOZOBIL® (ANORMed, Vancouver, Canada)), and pharmaceutically acceptable salts thereof:

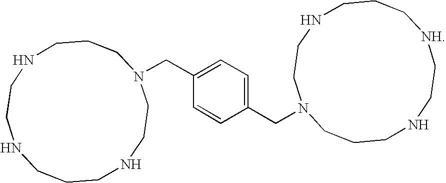

Other art-recognized CXCR4 antagonists useful in the methods of the invention are disclosed, for example, in U.S. Pat. Nos. 5,021,409; 6,001,826; 5,583,131; 5,698,546; 5,817,807 and 6,506,770 incorporated herein by reference, and in PCT publications WO 92/16494; WO 93/12096; WO 95/18808; WO 00/02870; WO 00/56729; WO 01/44229; WO 02/22600; WO 02/22599; WO 02/34745, WO 03/055876; WO 04/091518 and WO 04/093217, also incorporated by reference. Additional compounds that are CXCR4 antagonists are disclosed in U.S. application Ser. No. 10/823,494 filed 12 Apr. 2004 and Ser. No. 10/831,098 filed 22 Apr. 2004 and Ser. No. 11/012,002 filed 13 Dec. 2004, incorporated herein by reference. Other CXCR4 antagonists that may be used to practice the methods of the invention also include but are not limited to CTCE-0214; CTCE-9908; CP-1221 (linear peptides, cyclic peptides, natural amino-acids, unnatural amino acids, and peptidomimetic compounds); other peptide-based antagonists (see e.g., WO 01/85196; WO 00/09152, and WO 99/47158); anti-CXCR4 or anti-SDF-1 antibodies (e.g., WO 99/50461); T22 (Murkami et al., J. Exp. Med., (1997) 186: 1389-93), T140 and analogs; 4F-benzoyl-TN24003; KRH-1120; KRH-1636; KRH-2731; polyphemusin analogue; ALX40-4C (Doranz et al., J. Exp. Med., (1997), 186: 1395-1400; Donzella et al., Nat. Med., (1998) 4: 72-77); or those described in WO 01/85196; WO 99/50461; WO 01/94420; WO 03/090512, each of which is incorporated by reference herein. Methods for preparation of these substances can be found, for example, in J. Exp. Med., (1997), 186: 1189-1191.

### IV. Chemokine Antagonists

In certain aspects of the invention, a VDA is administered together with a chemokine antagonist. A particularly preferred chemokine antagonist is an antagonist of the chemokine Stromal Cell Derived Factor-1 (“SDF-1”, also known as CXCL12), the cognate ligand of CXCR4. This pleiotropic chemokine is constitutively or inducibly expressed in several organs including lung, liver, skin, and bone marrow (Ratajczak et al., Leukemia, 20: 191-1924 (2006). Moreover, SDF-1 is expressed in a large number of tumors and has been shown to play a role in angiogenesis (Ara et al., Blood, 105:3155-3161 (2005)). Inhibition of biological function of SDF-1 (e.g., by blocking SDF-1 binding to CXCR4) will therefore inhibit the formation of the vasculature necessary for tumor growth. Accordingly, in certain aspects of the invention, a VDA is administered together with an SDF-1 antagonist to thereby potentiate the effect of a VDA.

Any antagonist that interferes with the biological function of SDF-1 may be employed in the methods of the invention. In certain embodiments, the SDF-1 antagonist is a polypeptide which binds to SDF-1 and interferes with the binding of SDF-1 to its cognate receptor (e.g., CXCR4). Suitable polypeptides include, but are not limited to, antibodies or antigen binding fragments thereof. Suitable antibodies are monoclonal antibodies (e.g., human, humanized, or chimeric anti-SDF-1 antibodies) with binding specificity for SDF-1 (see, e.g., WO 08/018,641, which is incorporated by reference herein). In other embodiments, the SDF-1 antagonist is a peptide (see, e.g., U.S. Pat. No. 6,613,742). In other embodiments, the SDF-1 antagonist is an siRNA which cleaves SDF-1 mRNAs via RNA interference and prevents SDF-1 protein expression (see, e.g., US Patent Publication No. 2006/0019917, which is incorporated by reference herein) In certain embodiments, the SDF-1 antagonist is small molecule which binds to SDF-1 and interferes with the binding of SDF-1 to CXCR4 (see, e.g., U.S. Pat. No. 7,052,676, WO 04/058705, which is incorporated by reference herein).

### V. Preferred Dosage Ranges

### Two-Component Combination Therapy

In accordance with the present invention, improved, two-component chemotherapeutic regimens comprising a VDA (e.g., a combretastatin) and a CXCR4 antagonist (and/or SDF-1 antagonist) are provided for the treatment of cancer. The improved chemotherapeutic regimens can enhance efficacy for the treatment of neoplastic disease. For example, the present methods permit a clinician to administer a combretastatin compound, and a CXCR4 antagonist (and/or SDF-1 antagonist), at dosages which are significantly lower than those employed for the single agent. Preferred dosages suitable for administration of the compound of Formula I and combretastatin compounds in accordance with the invention are set forth herein below. Whether administered simultaneously or sequentially, the combretastatin compound and the at least one anticancer agent can be administered in any amount or by any route of administration effective for the modulation of tumor growth or metastasis, especially treatment of cancer as described herein.

In one exemplary embodiment, a combretastatin prodrug (e.g. CA4P or CA1P) is administered together with AMD3100. In a particularly preferred embodiment, a pharmaceutical composition comprising AMD3100 and CA1P are used to treat cancer in a subject, wherein the subject is human. In another preferred embodiment, a pharmaceutical composition comprising AMD3100 and CA4P are used to treat cancer in a subject, wherein the subject is human.

A suitable dose per day for each of the compounds, i.e., a CXCR4 or SDF-1 antagonist, and a VDA (e.g. a combretastatin), can be, individually, in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

Other suitable doses for the compounds of the invention include, for example, 0.1 mg/kg to about 100 mg/kg; from about 1 mg/kg to about 100 mg/kg; from about 5 mg/kg to about 50 mg/kg; from about 10 to about 25 mg/kg; about 10 mg/kg; about 15 mg/kg; about 20 mg/kg; about 25 mg/kg; about 30 mg/kg; about 40 mg/kg; about 50 mg/kg; about 60 mg/kg; about 70 mg/kg; about 80 mg/kg; about 90 mg/kg; and about 100 mg/kg. In a preferred embodiment, the VDA (e.g., a combretastatin agent) is administered at a dose ranging from between 45 mg/kg and 63 mg/kg.

### VI. Multi-Component Combination Therapy

In certain aspects, the combination therapy methods and pharmaceutical compositions of the invention may comprise other anticancer agents in addition to a VDA and/or CXCR or chemokine antagonist. As explained above, numerous types of anticancer agents are exemplary of those having applications in a combination therapy with the pharmaceutical compositions (e.g., AMD3100 and CA1P or CA4P) and methods of the present invention. Such classes of anticancer agents, and their preferred mechanisms of action, are described below:

1. Alkylating agent: a compound that donates an alkyl group to nucleotides. Alkylated DNA is unable to replicate itself and cell proliferation is stopped. Examples of such compounds include, but are not limited to, busulfan (Myleran®), coordination metal complexes (e.g. platinum coordination compounds such as carboplatin, oxaliplatin, and cisplatin), cyclophosphamide (Cytoxan®), dacarbazine, ifosfamide, lomustine, procarbazine, mechlorethamine (mustargen), and melphalan;

2. Bifunctional alkylating agent: a compound having two labile methanesulfonate groups that are attached to opposite ends of a four carbon alkyl chain. The methanesulfonate groups interact with, and cause damage to DNA in cancer cells, preventing their replication. Examples of such compounds include, without limitation, chlorambucil and melphalan;

3. Non-steroidal aromatase inhibitor: a compound that inhibits the enzyme aromatase, which is involved in estrogen production. Thus, blockage of aromatase results in the prevention of the production of estrogen. Examples of such compounds include anastrozole and exemstane;

4. Immunotherapeutic agent: an antibody or antibody fragment which targets cancer cells that produce proteins associated with malignancy. Exemplary immunotherapeutic agents include Herceptin® (Genentech, South San Francisco, Calif.) which targets HER2 or HER2/neu, which occurs in high numbers in about 25 percent to 30 percent of breast cancers; Erbitux® (ImClone Systems, New York, N.Y.) which targets the Epidermal Growth Factor Receptor (EGFR) in colon cancers; Avastin® (Genentech, South San Francisco, Calif.) which targets the Vascular Endothelial Growth Factor (VEGF) expressed by colon cancers; and rituximab (Rituxan®, Genentech, South San Francisco, Calif.) an anti-CD20 antibody which triggers apoptosis in B cell lymphomas. Additional immunotherapeutic agents include immunotoxins, wherein toxin molecules such as ricin, diphtheria toxin and pseudomonas toxins are conjugated to antibodies which recognize tumor specific antigens. Conjugation can be achieved biochemically or via recombinant DNA methods.

5. Nitrosurea compound: inhibits enzymes that are needed for DNA repair. These agents are able to travel to the brain so they are used to treat brain tumors, as well as non-Hodgkin's lymphomas, multiple myeloma, and malignant melanoma. Examples of nitrosureas include carmustine and lomustine;

6. Antimetabolite: a class of drugs that interfere with DNA and ribonucleic acid (RNA) synthesis. These agents are phase specific (S phase) and are used to treat chronic leukemias as well as tumors of breast, ovary and the gastrointestinal tract. Examples of antimetabolites include 5-fluorouracil, 6-thioguanine, 6-mercaptopurine, 5-azacytidine, cladribine, fludarabine, hydroxyurea, methotrexate, gemcitabine (GEMZAR®), cytarabine (cytosine arabinoside, Ara-C, Cytosar-U), and fludarabine.

7. Antitumor antibiotic: a compound having antimicrobial and cytotoxic activity. Such compounds also may interfere with DNA by chemically inhibiting enzymes and mitosis or altering cellular membranes. Examples include, but certainly are not limited to bleomycin, dactinomycin, daunorubicin, doxorubicin (Adriamycin), idarubicin, and the manumycins (e.g. Manumycins A, C, D, E, and G and their derivatives; see for example U.S. Pat. No. 5,444,087);

8. Mitotic inhibitor: a compound that can inhibit mitosis (e.g., tubulin binding compounds) or inhibit enzymes that prevent protein synthesis needed for reproduction of the cell. Examples of mitotic inhibitors include taxanes such as paclitaxel and docetaxel, epothilones, etoposide, vinblastine, vincristine, and vinorelbine.

9. Radiation therapy: includes but is not limited to X-rays or gamma rays which are delivered from either an externally supplied source such as a beam or by implantation of small radioactive sources.

10. Topoisomerase I inhibitors: agents which interfere with topoisomerase activity thereby inhibiting DNA replication. Such agents include, without limitation, CPT-11 and topotecan.

11. Hormonal therapy: includes, but is not limited to anti-estrogens, such as Tamoxifen, GnRH agonists, such as Lupron, and Progestin agents, such as Megace.

Naturally, other types of anticancer agents that function via a large variety of mechanisms have combination therapy application in the pharmaceutical compositions and methods of the present invention. Additional such agents include for example, leuocovorin, kinase inhibitors, such as Iressa and Flavopiridol, analogues of conventional chemotherapeutic agents such as taxane analogs and epothilone analogues, antiangiogenics such as matrix metalloproteinase inhibitors, and other VEGF inhibitors, such as Bevacizumab (Genentech, South San Francisco, Calif.). ZD6474 and SU6668. Retinoids such as Targretin can also be employed in the pharmaceutical compositions and methods of the invention. Signal transduction inhibitors which interfere with farnesyl transferase activity and chemotherapy resistance modulators, e.g., Valspodar can also be employed. Monoclonal antibodies such as C225 and anti-VEGFr antibodies can also be employed.

### VII. Pharmaceutical Compositions

As explained above, the present methods can, for example, be carried out using a single pharmaceutical composition comprising both a VDA and CXCR4 or SDF-1 antagonist when administration is to be simultaneous or sequential.

Pharmaceutical compositions employed in the methods of the invention include a compound (e.g., a VDA and/or CXC43 or SDF-1 antagonist) formulated with other ingredients, e.g., “pharmaceutically acceptable carriers”. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Other pharmaceutical carriers include, but are not limited to, antioxidants, preservatives, dyes, tablet-coating compositions, plasticizers, inert carriers, excipients, polymers, coating materials, osmotic barriers, devices and agents which slow or retard solubility, etc. Non-toxic pharmaceutically acceptable excipients that are suitable for the manufacture of tablets include, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. Suitable pharmaceutical carriers are described in “Remington's Pharmaceutical Sciences” by E. W. Martin.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

Suitable pharmaceutical compositions for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions. Such compositions may contain one or more agents selected from the group consisting of diluents, sweetening agents, flavoring agents, coloring agents and preserving agents in order to provide palatable preparations. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds of the invention may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds of the invention may also be administered in the form of suppositories for rectal or vaginal administration of the drug. These compositions can be prepared by mixing the drug with a suitable non-irritating excipient which is solid at ordinary temperatures but liquid at the rectal temperature or vaginal temperature and will therefore melt in the rectum or vagina to release

### VIII. Disease Indications

Diseases which can be treated in accordance with present invention include, but are not limited: Accelerated Phase Chronic Myelogenous Leukemia; Acute Erythroid Leukemia; Acute Lymphoblastic Leukemia; Acute Lymphoblastic Leukemia in Remission; Acute Lymphocytic Leukemia; Acute Monoblastic and Acute Monocytic Leukemia; Acute Myelogenous Leukemia; Acute Myeloid Leukemia; Adenocarcinoma; Adenocarcinoma of the Colon; Adenocarcinoma of the Esophagus; Adenocarcinoma of the Lung; Adenocarcinoma of the Pancreas; Adenocarcinoma of the Prostate; Adenocarcinoma of the Rectum; Adenocarcinoma of the Stomach; Adenoid Cystic Carcinoma of the Head and Neck; Adenosquamous Cell Lung Cancer; Adult Giant Cell Glioblastoma; Advanced Adult Primary Liver Cancer; Advanced Gastrointestinal Stromal Tumor; Advanced Non-Nasopharyngeal Head and Neck Carcinoma; Advanced NSCLC; Advanced Solid Tumors; Agnogenic Myeloid; Metaplasia; Anaplastic Astrocytoma; Anaplastic Oligodendroglioma; Anaplastic Thyroid Cancer; Astrocytoma; Atypical Chronic Myelogenous Leukemia; B-Cell Adult Acute Lymphoblastic Leukemia; Bladder Cancer; Blastic Phase Chronic Myelogenous Leukemia; Bone Metastases; Brain Tumor; Breast Cancer; Breast Cancer in Situ; Breast Neoplasms; Brenner Tumor; Bronchoalveolar Cell Lung Cancer; Cancer of the Fallopian Tube; Carcinoma, Squamous Cell; Central Nervous System Cancer; Cervix Neoplasms; Childhood Acute Lymphoblastic Leukemia; Childhood Acute Lymphoblastic Leukemia in Remission; Childhood Brain Tumor; Childhood Central Nervous System Germ Cell Tumor; Childhood Cerebellar Astrocytoma; Childhood Chronic Myelogenous Leukemia; Childhood Ependymoma; Childhood Malignat Germ Cell Tumor; Childhood Oligodendroglioma; Childhood Soft Tissue Sarcoma; Chordoma; Chronic Eosinophilic Leukemia (CEL); Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Congenital Fibrosarcoma; Dermatofibrosarcoma; Dermatoftbrosarcoma Protuberans (DFSP); Desmoid Tumor; Endometrial Adenocarcinoma; Endometrial Adenosquamous Cell; Eosinophilia; Esophageal Cancer; Epidemic Kaposi's Sarcoma; Epithelial Mesothelioma; Esophageal Cancer; Esophagogastric Cancer; Essential Thrombocythemia; Ewing's Family of Tumors; Extensive Stage Small Cell Lung Cancer; Extrahepatic Bile Duct Cancer; Fallopian Tube Cancer; Familiar Hypereosinophilia; Fibrosarcoma; Follicular Thyroid Cancer; Gallbladder Cancer; Gastric Adenocarcinoma; Gastric Cancer; Gastroinstestinal Cancer; Gastrinoma; Gastrointestinal Carcinoid; Gastrointestinal Neoplasm; Gastrointestinal Stromal Tumor; Giant Cell Glioblastoma; Glioblastoma; Glioma; Glioblastoma Multiforme; Gliosarcoma; Grade I Meningioma; Grade II Meningioma; Grade III Meningioma; Head and Neck Cancer; Head and Neck Neoplasms; Hematopoietic and Lymphoid Cancer, Hepatocellular Carcinoma; High-Grade Childhood Cerebral Astrocytoma; Hypereosinophilic Syndrome; Hypopharyngeal Cancer; Idiopathic Pulmonary Fibrosis; Inflammatory Myofibroblastic Tumor; Inoperable Locally Advanced Squamous Cell Carcinoma of Head and Neck; Insulinoma; Intraductal Breast Carcinoma; Islet Cell Carcinoma; Kidney and Urinary Cancer; L1 Adult Acute Lymphoblastic Leukemia; L2 Adult Acute Lymphoblastic Leukemia; Large Cell Lung Cancer; Laryngeal Cancer; Leukemia, Lymphocytic, Acute L2; Leukemia, Myeloid, Chronic; Leukemia, Myeloid, Chronic Phase; Lip and Oral Cavity Cancer; Lip Cancer; Liver Cancer; Liver Dysfunction and Neoplasm; Localized Unresectable Adult Primary Liver Cancer; Low-Grade Childhood Cerebral Astrocytoma; Lymphoid Blastic Phase of Chronic Myeloid Leukemia; Lung Adenocarcinoma With Bronchiole-Alveolar Feature; Lung Cancer; Male Breast Cancer; Malignant Fibrous Histiocytoma; Malignant Melanoma; Mastocytosis; Medullary Thyrod Cancer; Melanoma; Meningeal Tumors; Meningeal Hemangiopericytoma; Meningioma; Meningioma; Meningioma; Mesothelioma; Metastatic Cancer; Metastatic Solid Tumors; Metastatic Colorectal Cancer; Metastatic Gastrointestinal Carcinoid Tumor; Metastatic Pancreatic Carcinoma; Mixed Gliomas; Multiple Myeloma; Musculoskeletal Tumors; Myelodysplastic Syndrome; Myelogenous Leukemia, Acute; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Nasopharyngeal Cancer; Nasopharyngeal Carcinoma; Neoplasms; Neuroblastoma; Neurofibrosarcoma; Non-B Childhood Acute Lymphoblastic Leukemia; Non-Metastatic (T2-T4, N0-N3, MO; Stages II and III) and Histologically-Confirmed Intestinal GC; Non-Metastatic Prostate Cancer; Nonresectable Adrenocortical Carcinoma; Non-Small Cell Lung Cancer; Nose Cancer; Oligodendroglioma; Oligodendroglial Tumors; Oral Cancer; Oropharyngeal Cancer; Osteosarcoma; Ovarian Cancer; Ovarian Epithelial Cancer; Ovarian Germ Cell Tumor; Ovarian Low Malignant Potential Tumor; Ovarian Neoplasms; Pancreatic Cancer; Papillary Thyroid Cancer; Pelvic Neoplasms; Peritoneal Cavity Cancer; Peritoneal Carcinoma; Peritoneal Neoplasms; Pharynx Cancer; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Pneumonic-Type Adenocarcinoma (P-ADC); Polycythemia Vera; Pulmonary Fibrosis; Primary Hepatocellular Carcinoma; Primary Liver Cancer; Prostate Cancer; Prostate Cancer, Antigen Independent; Rectal Cancer; Recurrent Adult Brain Tumor; Recurrent Adult Soft Tissue Sarcoma; Recurrent Adult Primary Liver Cancer; Recurrent Breast Cancer; Recurrent Cervical Cancer; Recurrent Colon Cancer; Recurrent Endometrial Cancer, Recurrent Esophageal Cancer; Recurrent Gastric Cancer; Recurrent Glioblastoma; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; Recurrent Pancreatic Cancer; Recurrent Prostate Cancer; Recurrent Rectal Cancer; Recurrent Salivary Gland Cancer; Recurrent Skin Cancer; Recurrent Small Cell Lung Cancer; Recurrent Tumors of the Ewing's Family; Recurrent Uterine Sarcoma; Refractory Germ Cell Tumors Expressing EGRF; Relapsing Chronic Myelogenous Leukemia; Renal Cell Cancer; Renal Cell Carcinoma; Renal Papillary Carcinoma; Rhabdomyosarcomas; Salivary Gland Adenoid Cystic Carcinoma; Sarcoma; Sarcomatous Mesothelioma; Skin Cancer; Small Cell Lung Cancer; Soft Tissue Sarcoma; Squamous Cell Carcinoma; Squamous Cell Carcinoma of the Esophagus; Squamous Cell Carcinoma of the Head and Neck; Squamous Cell Carcinoma of the Skin; Squamous Cell Lung Cancer; Stage II Esophageal Cancer; Stage III Esophageal Cancer, Stage II Melanoma; Stage II Merkel Cell Carcinoma; Stage III Adult Soft Tissue Sarcoma; Stage III Esophageal Cancer; Stage III Merkel Cell Carcinoma; Stage III Ovarian Epithelial Cancer; Stage III Pancreatic Cancer; Stage III Salivary Gland Cancer; Stage IIIB Breast Cancer; Stage IIIC Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Systemic Mastocytosis; Synovial Sarcoma; T-lymphoma; T-Cell Childhood Acute Lymphoblastic Leukemia; Testicular Cancer; Thorax and Respiratory Cancer; Throat Cancer; Thyroid Cancer; Transitional Cell Cancer of the Renal Pelvis and Ureter; Transitional Cell Carcinoma of the Bladder; Tubal Carcinoma; Unresectable or Metastatic Malignant Gastrointestinal Stromal Tumor (GIST); Unspecified Childhood Solid Tumor; Unspecified Adult Solid Tumor; Untreated Childhood Brain Stem Glioma; Urethral Cancer; Uterine Carcinosarcoma, and Uterine Sarcoma.

### IX. Methods of Administration

As explained above, the present invention is directed towards methods for modulating tumor growth and metastasis comprising, inter alia, the administration of a VDA and a CXCR4 or SDF-1 antagonist. The agents of the invention can be administered separately (e.g. formulated and administered separately), or in combination as a pharmaceutical composition of the present invention. Administration can be achieved by any suitable route, such as parenterally, transmucosally, e.g., orally, nasally, or rectally, or transdermally. Preferably, administration is parenteral, e.g., via intravenous injection. Alternative means of administration also include, but are not limited to, intra-arteriole, intramuscular, intradermal, subcutaneous, intraperitoneal, intraventricular, and intracranial administration/or by injection into the tumor(s) being treated or into tissues surrounding the tumor(s).

The pharmaceutical composition may be employed in any suitable pharmaceutical formulation, as described above, including in a vesicle, such as a liposome [see Langer, Science 249:1527-1533 (1990); Treat et al., in Liposomes in the Therapy of Infectious Disease and Cancer, Lopez-Berestein and Fidler (eds.), Liss: New York, pp. 317-327, see generally, ibid] Preferably, administration of liposomes containing the agents of the invention is parenteral, e.g., via intravenous injection, but also may include, without limitation, intra-arteriole, intramuscular, intradermal, subcutaneous, intraperitoneal, intraventricular, and intracranial administration, or by injection into the tumor(s) being treated or into tissues surrounding the tumor(s).

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)]. In another embodiment, polymeric materials can be used [see Medical Applications of Controlled Release, Langer and Wise (eds.)/CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target tissues of the animal, thus requiring only a fraction of the systemic dose [see, e.g., Goodson, in Medical Applications of Controlled Release, supra, vol. 2, pp. 115-138 (1984).]. In particular, a controlled release device can be introduced into an animal in proximity of the site of inappropriate immune activation or a tumor. Other controlled release systems are discussed in the review by Langer [Science 249:1527-1533 (1990)].

A controlled release formulation can be pulsed, delayed, extended, slow, steady, immediate, rapid, fast, etc. It can comprise one or more release formulations, e.g. extended- and immediate-release components. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of a compound of the invention.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157 3 Mar. 1994). For example, a solution or suspension of a compound of the invention in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of the invention may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C18 fatty alcohols such as lauryl alcohol or cetyl alcohol, saturated or unsaturated C8-C18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C8-C18 fatty alcohols, saturated or unsaturated 08-C18 fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

### X. Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London. and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which can not be considered pharmaceutically acceptable, can be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

### EQUIVALENTS

Those skilled in the art will recognize, or be able to ascertain using no more than routine experimentation, many equivalents to the specific embodiments of the invention described herein. Such equivalents are intended to be encompassed by the following claims.

The following examples are provided to illustrate embodiments of the invention. They are not intended to limit the invention in any way.

### EXAMPLES

### Example 1

### Synergistic Enhancement of CEP Levels in Non-Tumor Bearing Mice Following VDA+CXCR4 Antagonist Combination Therapy

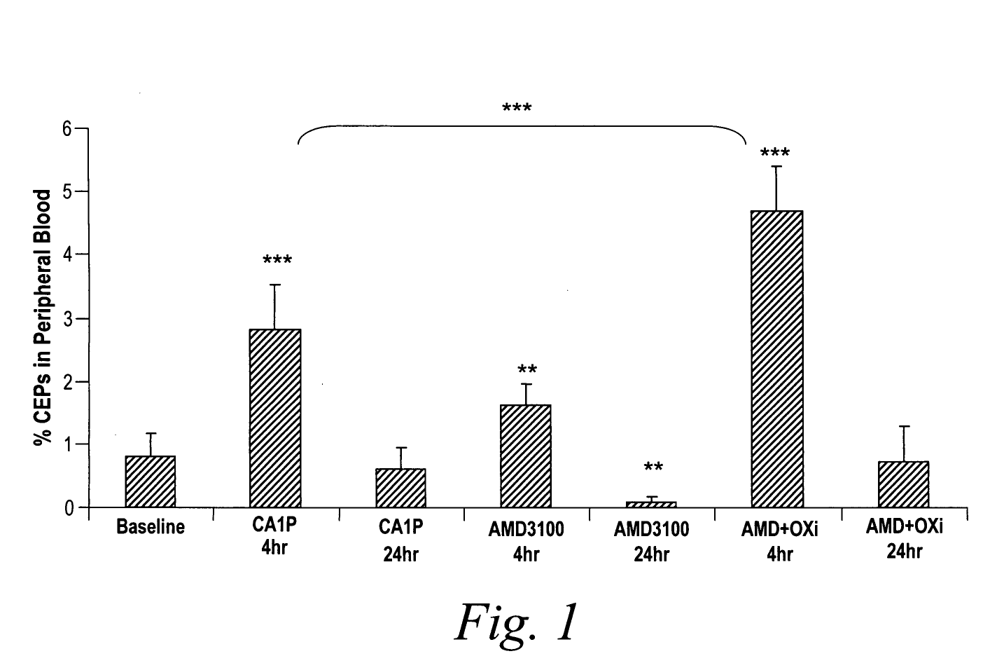

Non-tumor bearing Balb/c mice (8-10 weeks old) were treated with one of the following agents: (i) 5 mg/kg of AMD-3100, a specific CXCR-4 antagonist; (ii) 100 mg/kg of CA1P, a VDA; or (iii) a combination of AMD-3100 and CA1P. At either 4 hours or 24 hours post-treatment, mice were bled from the retro-orbital sinus to evaluate levels of CEPs by flow cytometry. The results in FIG. 1 demonstrate that both CXCR-4 antagonists and CA1P cause rapid and significant increases in CEP levels in peripheral blood. CEP levels following the combination treatment AMD-3100 and CA1P were significantly higher than following treatment with either agent alone.

### Example 2

### Synergistic Enhancement of Anti-Tumor Effects in Tumor Bearing Mice following VDA+CXCR4 Antagonist Combination Therapy

### (a) Synergistic Enhancement of an Anti-Tumor Effect in a Lung Cancer Model

To assess the incorporation of bone marrow proangiogenic cells to the tumor site, Lewis-Lung Carcinoma (LLC) cells were implanted into the flanks of lethally irradiated C57Bl mice that were previously transplanted with GFP+bone marrow tagged cells. When tumors reached 500 mm³, treatment with CA1P, AMD-3100 or the combination of the two was initiated. AMD-3100 was given in a concentration of 5 mg/kg for 3 sequential days. On day 4, mice were sacrificed and tumors removed for the evaluation of bone marrow derived cell (GFP+ cells) invasion and incorporation into tumor blood vessels (CD31+ cells). Tumors were also evaluated for necrosis by green fluorescence, as necrotic tissue is auto-fluorescent under GFP (fluorescent) channel. The results demonstrated a greater tumor necrosis induced by the combination treatment, exceeding 70%. In addition, AMD-3100 blocked the bone marrow cell homing and incorporation into the tumor blood vessels, assessed by a decrease in GFP+ cells in the tumor site, none of which were co-localized with CD31+ cells.

The above analysis demonstrated that the CA1P treatment group recruits bone marrow derived proangiogenic cells to tumors, some of which are incorporated into tumor blood vessels as evidenced by co-localization of GFP+ and CD31+ staining. AMD-3100 did not reveal a significant change in the presence of bone marrow cells at the tumor site in comparison to untreated control group. However, the combination of AMD-3100 and CA1P revealed a complete clearance of GFP+ bone marrow cells from the tumor site with no sign for their incorporation to tumor blood vessels. Moreover, enhanced tumor necrosis and tumor cell death was observed in the combination treatment strategy. Accordingly, although AMD-3100 and CA1P generate elevated levels of CEPs following administration (see Example 1), these cells do not home to the tumor, nor are they retained at the tumor site, as indicated by the profound lack of GFP+ bone marrow cells in the tumor of mice treated with the combination of these agents. Taken together, the antiangiogenic effect of AMD-3100 in the context of VDA treatment may stem from the inhibition of CXCR4+ cell recruitment and retention in the tumor site, despite the ability of this drug to induce mobilization of CEPs.

### (b) Synergistic Enhancement of an Anti-tumor Effect in a Melanoma Tumor Model

MeWo human melanoma cells were implanted subdermally in nude mice. When tumors reached 500 mm³, treatment with CA1P, AMD-3100 or the combination of the two drugs was initiated. CA1P was given as a single injection and AMD-3100 was given in a concentration of 5 mg/kg for 3 sequential days. On day 4, mice were sacrificed and tumors harvested for evaluation of the expression of human SDF-1 and mouse CXCR4, as well as tumor necrosis, hypoxia and vessel perfusion.

Longitudinal tumor sections were stained for either SDF-1 or mouse CXCR4 expression. The results of this analysis indicated that CA1P treatment causes upregulation in human SDF-1 levels in tumors and invasion of mouse CXCR4+ cells to the tumor site, respectively. The combination of CA1P, followed by AMD-3100, blocked the invasion of CXCR4+ cells to the tumor site.

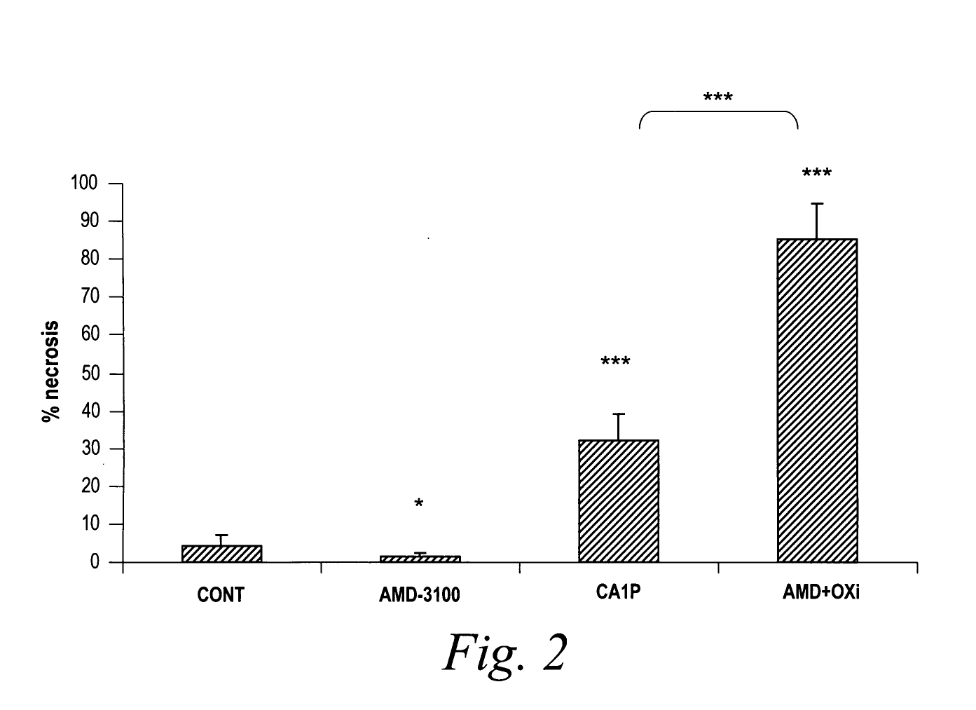

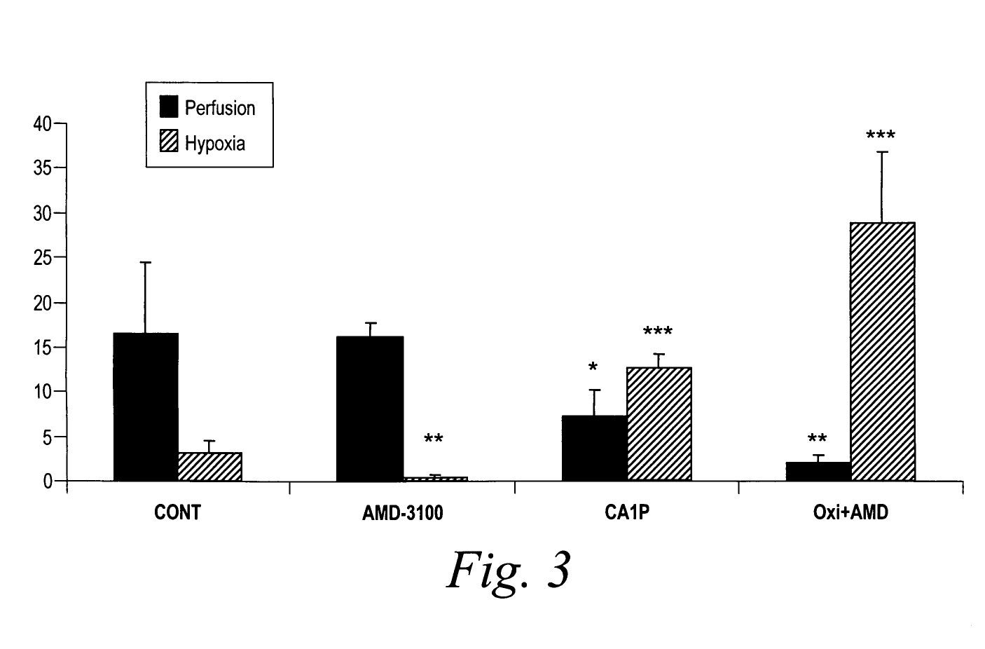

A greater degree of tumor necrosis, increase in tumor hypoxia, and a decrease in tumor perfusion was also revealed following drug combination treatment. FIG. 2 provides a statistical quantification of the relative necrosis area in all tumors from treated and untreated mice. Hypoxic tumor area and perfused tumor area was also measured for all treated and untreated tumors using pimonidazole and Hoechst staining respectively (see FIG. 3).

Overall, the results demonstrate a substantial tumor necrosis (˜30%) with increased hypoxia (˜12%) and reduced perfusion (˜%7) in CA1P treated mice in comparison to control untreated mice. Interestingly, the combination of CA1P and AMD-3100 synergized the treatment effect by causing a substantial increase in tumor necrosis (˜90%) and massive tumor hypoxia (˜30%) with almost a complete loss of tumor perfusion (˜2%). Taken together, these results demonstrate that the combination of AMD-3100 and CA1P provides a synergistic enhancement of treatment efficacy in comparison to the treatment with either AMD-3100 and CA1P alone.

### Example 3

### Synergistic Enhancement of Anti-Tumor Growth In Vivo Following VDA+CXCR4 Antagonist Combination Therapy

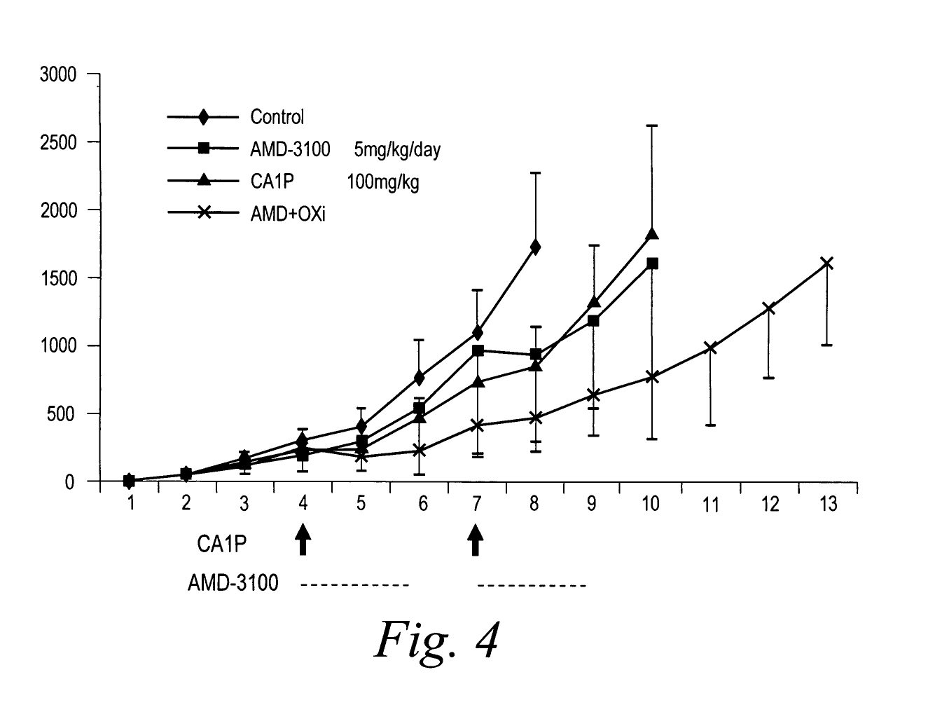

Since 70% necrosis was observed in the combination treatment of CA1P and AMD-3100, a long-term experiment was performed on mice bearing 200 mm³ MeWo human melanoma tumors. Two million MeWo human melanoma cells were implanted subdermally in 6-8 week old nude mice. When tumors reached 200 mm³, mice were administered a bolus injection of CA1P (100 mg/kg) once every 3 weeks, AMD-3100 (5 mg/kg/day) for a period of 2 weeks with one week drug-free break, or a combination of the two drugs schedule was initiated. Tumor volumes were assessed regularly. The results in FIG. 4 demonstrate a substantial inhibition of tumor growth in mice receiving the combination treatment over either of the monotherapy groups.

### Example 4

### VDA Treatment Results in Elevated Levels of SDF-1

To investigate the possibility that systemic induction of SDF-1 is involved in the acute mobilization of CEPs by VDA treatment, plasma from non-tumor or 500 mm³ MeWo human melanoma bearing nude mice obtained 4 hours after treatment with CA1P was analyzed for human and mouse SDF-1 using specific ELISAs. From this analysis, it was determined that an acute 2 fold increase in SDF-1 occurs in both tumor-free and tumor-bearing nude mice following CA1P treatment. These results show that SDF-1, which is known to mediate mobilization of CEPs, is acutely increased by VDA treatment and that the SDF-1 is derived from normal tissue rather than from the tumor. Furthermore, clinical data from 12 cancer patients with advanced solid tumors that were treated with a VDA (CA4P) showed that circulating SDF-1 plasma levels, as well as the number of CD133 and CD34 positive cells, were substantially increased 4 hours after CA4P treatment. Thus, there is a rapid host proangiogenic reaction in response to VDA treatment similar to that observed in mice. Accordingly, an SDF-1 antagonist (e.g., an SDF-1 antibody), when administered in combination with a VDA, will potentiate the anti-angiogenic effect of the VDA and hence provide an improved anti-tumor therapy.

1. A method for producing an anti-tumor effect in a subject suffering from cancer or a tumor, the method comprising administering to the patient a Vascular Disrupting Agent (VDA) and a CXCR antagonist in amounts effective therefor.

2. A method for preventing tumor regrowth in a subject suffering from cancer or a tumor, the method comprising administering to the patient a Vascular Disrupting Agent (VDA) and a CXCR antagonist in amounts effective therefor.

3. A method for inhibiting tumor-associated angiogenesis in a subject that is treated with a VDA, the method comprising administering to the patient a CXCR antagonist in amounts effective therefor.

4. A method for inhibiting homing and retention of circulating endothelial progenitor (CEP) cells or other proangiogenic cells to the tumor of a subject that is treated with a VDA, the method comprising administering to the patient a CXCR antagonist in amounts effective therefor.

5. The method of claim 1, wherein the CXCR antagonist is a CXCR4 antagonist.

6. The method of claim 1, wherein the VDA is a combretastatin agent.

7. The method of claim 5, wherein the CXCR4 antagonist is a compound of Formula I or a pharmaceutically acceptable salt or metal complex thereof:
Z-R—Ar—R′—Y (I)
wherein
Z and Y are each, independently, a cyclic polyamine moiety having a total of 9 to 24 atoms and from 2 to 6 optionally substituted nitrogens spaced by two or more optionally substituted carbon atoms from each other, and which may optionally comprise a fused aromatic or heteroaromatic ring;
R and R′ are each, independently, selected from the group consisting of straight, branched, or cyclic C₁₋₆-alkyl groups; and
Ar is an aromatic or heteroaromatic ring, optionally substituted at single or multiple positions with electron-donating or electron-withdrawing groups.

8. The method of claim 6, wherein the combretastatin agent is a compound of Formula II: 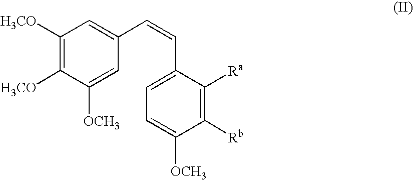
or a pharmaceutically acceptable salt thereof,
wherein
R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and
R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

9. The method of claim 6, wherein the combretastatin agent is a compound of Formula IIb: 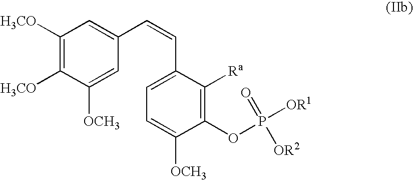
wherein R^{a} is H or OP(O)(OR³)OR⁴; and
OR¹, OR², OR³ and OR⁴ are each, independently, H, —O⁻QH⁺ or —O⁻M⁺, wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

10. The method of claim 8 wherein the compound of Formula IIb is administered at a dose ranging from between 45 mg/kg and 63 mg/kg.

11. The method of claim 9, wherein, for Formula IIb, R³ is H or OP(O)(OR³)OR⁴, and R¹, R², R³ and R⁴ are each, independently, an aliphatic organic amine, alkali metals, transition metal, heteroarylene, heterocyclyl, nucleoside, nucleotide, alkaloid, amino sugar, amino nitrile, or nitrogenous antibiotic.

12. The method of claim 9, wherein, for Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

13. The method of claim 9, wherein Formula IIb is represented by a compound of Formula III: 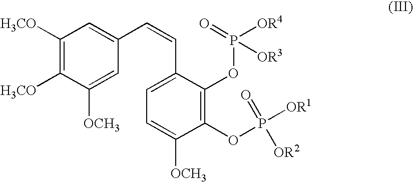
and pharmaceutically acceptable salts thereof.

14. The method of claim 7, wherein, for Formula I, Z and Y are each, independently, a cyclic polyamine moiety having a total of 14 to 20 atoms and from 3 to 6 optionally substituted amino nitrogens spaced by two or more optionally substituted carbon atoms from each other.

15. The method of claim 7, wherein, for Formula I, Ar is phenyl.

16. The method of claim 7, wherein, for Formula I, R and R′ are CH₂.

17. The method of claim 7, wherein Formula I is represented by 1,1′-[1,3-phenylenebis(methylene)]-bis-1,4,8,11-tetra-azacyclotetradecane; 1,1′-[1,4-phenylenebis(methylene)]-bis-1,4,8,11-tetra-azacyclotetradecane; 1,1′-[3,3′-biphenylene-bis-(methylene)]-bis-1,4,8,11-tetraazacyclotetradecane; 11,11′-[1,4-phenylene-bis-(methylene)]-bis-1,4,7,11-tetraazacyclotetradecane; 1,11′-[1,4-phenylene-bis-(methylene)]-1,4,8,11-tetraazacyclotetradecane-1,4,7,11-tetraazacyclotetradecane; 1,1′-[2,6-pyridine-bis-(methylene)]-bis-1,4,8,11-tetraazacyclotetradecane; 1,1-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[5-nitro-1,3-phenylenebis(methylene)]bis-1,4,8,11-tetraazacyclotetradecane; 1,1′-[2,4,5,6-tetrachloro-1,3-phenyleneis(methylene)]bis-1,4,8,11-tetraazacyclotetradecane; 1,1′-[2,3,5,6-tetra-fluoro-1,4-phenylenebis(methylene)]bis-1,4,8,11-tetraazacyclotetradecane; 1,1′-[1,4-naphthylene-bis-(methylene)]bis-1,4,8,11-tetraazacyclotetradecane; 1,1′-[1,3-phenylenebis-(methylene)]bis-1,5,9-triazacyclododecane; 1,1′-[1,4-phenylene-bis-(methylene)]-1,5,9-triazacyclododecane; 1,1′-[3,3′-biphenylene-bis-(methylene)]-bis-1,4,8,11-tetraazacyclotetradecane; 1,1′-[2,6-pyridine-bis-(methylene)]-bis-1,4,8,11-tetraazacyclotetradecane; 1,1′-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[2,5-dimethyl-1,4-phenylenebis-(methylene)]-bis-1,4,8,11-tetraazacyclotetradecane; 1,1′-[2,5-dichloro-1,4-phenylenebis-(methylene)]-bis-1,4,8,11-tetraazacyclotetradecane; 1,1′-[2-bromo-1,4-phenylenebis-(methylene)]-bis-1,4,8,11-tetraazacyclotetradecane; or 1,1′-[6-phenyl-2,4-pyridinebis-(methylene)]-bis-1,4,8,11-tetraazacyclotetradecane; and pharmaceutically acceptable salts thereof.

18. The method of claim 1, wherein the compounds are simultaneously or sequentially administered.

19. The method of claim 1, wherein said cancer is selected from the group consisting of ovarian cancer, fallopian tube cancer, cervical cancer, breast cancer, lung cancer, melanoma, and primary cancer of the peritoneum.

20. The method of claim 19, wherein said tumor is a solid tumor selected from the group consisting of a melanoma, an ovarian tumor, a cervical tumor, a breast tumor, small cell lung tumor, a non-small cell lung tumor, a fallopian tube tumor, and a primary tumor of the peritoneum.

21. A method of treating a tumor in a subject in need thereof by administering to the subject a pharmaceutical composition comprising a compound of the Formula I and a compound of the Formula II or IIb wherein the compound of Formula I is administered first followed by administration of a compound of Formula II or IIb.

22. A method of treating a tumor in a subject in need thereof by administering to the subject a pharmaceutical composition comprising a compound of the Formula I and a compound of the Formula II or IIb, wherein the compound of Formula II or IIb is administered first followed by administration of a compound of Formula I.

23. A method of treating a tumor in a subject in need thereof by administering to the subject a pharmaceutical composition comprising a compound of the Formula I and a compound of the Formula II or IIb, wherein the compound of Formula I and the compound of Formula II or IIb are administered simultaneously.

24. A method of treating a tumor in a subject in need thereof by administering to the subject a pharmaceutical composition comprising a AMD3100 and CA1P.

25. A method of treating a tumor in a subject in need thereof by administering to the subject a pharmaceutical composition comprising AMD3100 and CA4P.

26. The method of claim 1, wherein the subject is a mammal.

27. The method of claim 26, wherein the mammal is a human.

28. A pharmaceutical composition for producing an anti-tumor effect in a subject suffering from cancer or a tumor, comprising a Vascular Disrupting Agent (VDA) and a CXCR antagonist in amounts effective therefore in a pharmaceutical carrier.

29. The composition of claim 28, wherein the CXCR antagonist is a CXCR4 antagonist.

30. The composition of claim 28, wherein the VDA is a combretastatin agent.

31. (canceled)

32. (canceled)

33. The composition of claim 29, wherein the CXCR4 antagonist is a compound of Formula I or a pharmaceutically acceptable salt or metal complex thereof:
Z-R—Ar—R′—Y (I)
wherein
Z and Y are each, independently, a cyclic polyamine moiety having a total of 9 to 24 atoms and from 2 to 6 optionally substituted nitrogens spaced by two or more optionally substituted carbon atoms from each other, and which may optionally comprise a fused aromatic or heteroaromatic ring;
R and R′ are each, independently, selected from the group consisting of straight, branched, or cyclic C₁₋₆-alkyl groups; and
Ar is an aromatic or heteroaromatic ring, optionally substituted at single or multiple positions with electron-donating or electron-withdrawing groups.

34. The composition of claim 30, wherein the combretastatin agent is a compound of Formula II: 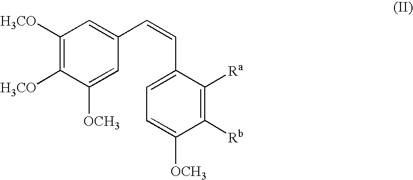
or a pharmaceutically acceptable salt thereof,
wherein
R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and
R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

35. The composition of claim 30, wherein the combretastatin agent is a compound of Formula IIb: 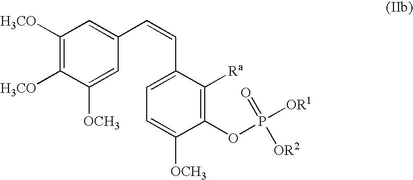
wherein R^{a} is H or OP(O)(OR³)OR⁴; and
OR¹, OR², OR³ and OR⁴ are each, independently, H, —O⁻QH⁺ or —O⁻M⁺, wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

36. The composition of claim 35, wherein the compound of Formula IIb is administered at a dose ranging from between 45 mg/kg and 63 mg/kg.

37. The composition of claim 35, wherein, for Formula IIb, R³ is H or OP(O)(OR³)OR⁴, and R¹, R², R³ and R⁴ are each, independently, an aliphatic organic amine, alkali metals, transition metal, heteroarylene, heterocyclyl, nucleoside, nucleotide, alkaloid, amino sugar, amino nitrile, or nitrogenous antibiotic.

38. The composition of claim 35, wherein, for Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

39. The composition of claim 35, wherein Formula IIb is represented by a compound of Formula III: 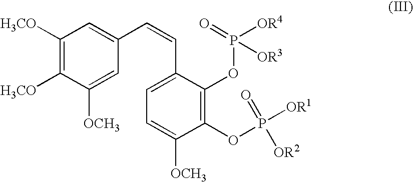
and pharmaceutically acceptable salts thereof.

40. The composition of claim 33, wherein, for Formula I, Z and Y are each, independently, a cyclic polyamine moiety having a total of 14 to 20 atoms and from 3 to 6 optionally substituted amino nitrogens spaced by two or more optionally substituted carbon atoms from each other.

41. The composition of claim 33, wherein, for Formula I, Ar is phenyl.

42. The composition of claim 33, wherein, for Formula I, R and R′ are CH₂.

43. The composition of claim 33, wherein Formula I is represented by 1,1′-[1,3-phenylenebis(methylene)]-bis-1,4,8,1,1-tetra-azacyclotetradecane; 1,1′-[1,4-phenylenebis(methylene)]-bis-1,4,8,11-tetra-azacyclotetradecane; 1,1′-[3,3′-biphenylene-bis-(methylene)]-bis-1,4,8,11-tetraazacyclotetradecane; 11,11′-[1,4-phenylene-bis-(methylene)]-bis-1,4,7,11-tetraazacyclotetradecane; 1,11′-[1,4-phenylene-bis-(methylene)]-1,4,8,11-tetraazacyclotetradecane-1,4,7,11-tetraazacyclotetradecane; 1,1′-[2,6-pyridine-bis-(methylene)]-bis-1,4,8,11-tetraazacyclotetradecane; 1,1-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[5-nitro-1,3-phenylenebis(methylene)]bis-1,4,8,11-tetraazacyclotetradecane; 1,1′-[2,4,5,6-tetrachloro-1,3-phenyleneis(methylene)]bis-1,4,8,11-tetraazacyclotetradecane; 1,1′-[2,3,5,6-tetra-fluoro-1,4-phenylenebis(methylene)]bis-1,4,8,11-tetraazacyclotetradecane; 1,1′-[1,4-naphthylene-bis-(methylene)]bis-1,4,8,11-tetraazacyclotetradecane; 1,1′-[1,3-phenylenebis-(methylene)]bis-1,5,9-triazacyclododecane; 1,1′-[1,4-phenylene-bis-(methylene)]-1,5,9-triazacyclododecane; 1,1′-[3,3′-biphenylene-bis-(methylene)]-bis-1,4,8,11-tetraazacyclotetradecane; 1,1′-[2,6-pyridine-bis-(methylene)]-bis-1,4,8,11-tetraazacyclotetradecane; 1,1′-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1,1′-[2,5-dimethyl-1,4-phenylenebis-(methylene)]-bis-1,4,8,11-tetraazacyclotetradecane; 1,1′-[2,5-dichloro-1,4-phenylenebis-(methylene)]-bis-1,4,8,11-tetraazacyclotetradecane; 1,1′-[2-bromo-1,4-phenylenebis-(methylene)]-bis-1,4,8,11-tetraazacyclotetradecane; or 1,1′-[6-phenyl-2,4-pyridinebis-(methylene)]-bis-1,4,8,11-tetraazacyclotetradecane; and pharmaceutically acceptable salts thereof.

44. The composition of claim 28, said pharmaceutical composition comprising AMD3100 and CA1P.

45. The composition of claim 28, said pharmaceutical composition comprising AMD3100 and CA4P.

46. A method for producing an anti-tumor effect in a subject suffering from cancer or a tumor, the method comprising administering to the patient a VDA and a SDF-1 antagonist in amounts effective therefor.

47. A method for preventing tumor regrowth in a subject suffering from cancer or a tumor, the method comprising administering to the patient a VDA and a SDF-1 antagonist in amounts effective therefor.

48. A method for inhibiting tumor-associated angiogenesis in a subject that is treated with a VDA, the method comprising administering to the patient a SDF-1 antagonist in amounts effective therefor.

49. A method for inhibiting homing and retention of circulating endothelial progenitor (CEP) cells or other proangiogenic cells to the tumor of a subject that is treated with a VDA, the method comprising administering to the patient a SDF-1 antagonist in amounts effective therefor.

50. The method of claim 46, wherein the VDA is a combretastatin agent.

51. The method of claim 50, wherein the combretastatin agent is a compound of Formula II: 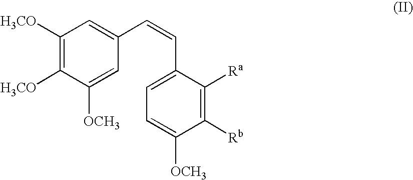
or a pharmaceutically acceptable salt thereof,
wherein
R^{a} is H, phosphate, phosphate ester, phosphonate, phosphoramidate monoester, phosphoramidate diester, cyclic phosphoramidate, phosphordiamidate, cyclic phosphorodiamidate, phosphonamidate or amino acid acyl; and
R^{b} is phosphate, phosphate ester, phosphonate, phosphoramidate monoester, phosphoramidate diester, cyclic phosphoramidate, phosphordiamidate, cyclic phosphorodiamidate, phosphonamidate or amino acid acyl.

52. The method of claim 50, wherein the combretastatin agent is a compound of Formula IIb: 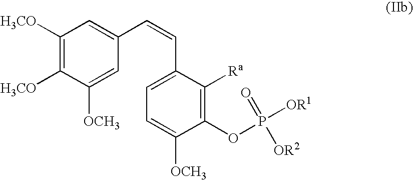
wherein R^{a} is H or OP(O)(OR³)OR⁴; and
OR¹, OR², OR³ and OR⁴ are each, independently, H, —O⁻QH⁺ or —O⁻M⁺, wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

53. The method of claim 52, wherein, for Formula IIb, R³ is H or OP(O)(OR³)OR⁴, and R¹, R², R³ and R⁴ are each, independently, an aliphatic organic amine, alkali metals, transition metal, heteroarylene, heterocyclyl, nucleoside, nucleotide, alkaloid, amino sugar, amino nitrile, or nitrogenous antibiotic.

54. The method of claim 52, wherein, for Formula IIb, R¹, R², R³ and R⁴ are each, independently, Na, TRIS, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

55. The method of claim 52, wherein Formula IIb is represented by a compound of Formula III: 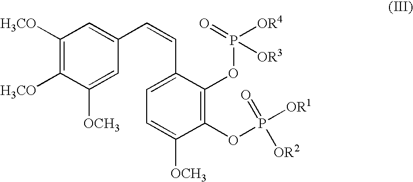
and pharmaceutically acceptable salts thereof.

56. A pharmaceutical composition for producing an anti-tumor effect in a subject suffering from cancer or a tumor, comprising a VDA and a SDF-1 antagonist in amounts effective therefore in a pharmaceutical carrier.
